(19) United States
(12) Patent Application Publication
(10) Pub. No.: US 2022/0031845 A1
(43) Pub. Date: Feb. 3, 2022

(54) DILUENTS FOR COMPOSITIONS OF CANNABINOIDS AND USES THEREOF

(71) Applicant: KANABO RESEARCH LTD., Tel Aviv (IL)

(72) Inventors: Avihu TAMIR, Tel Aviv (IL); Michael ADDA, Kfar Saba (IL)

(21) Appl. No.: 17/502,468

(22) Filed: Oct. 15, 2021

Related U.S. Application Data
(63) Continuation-in-part of application No. PCT/IL2020/050416, filed on Apr. 6, 2020.

(30) Foreign Application Priority Data
Apr. 18, 2019 (IL) 266151

(51) Int. Cl. A61K 47/10; A61K 9/00; A61K 31/05; A61K 31/465; A61P 25/34; A61M 16/00

(52) U.S. Cl. CPC A61K 47/10; A61K 9/0073; A61M 16/021; A61K 31/465; A61P 25/34; A61K 31/05

(57) ABSTRACT

The present invention provides a stock compositions comprising a combinations of terpenes, that, when used as diluents in vaporizable formulations comprising cannabinoids, confer administration of reproducible constant dose of the formulations and its components such as cannabinoids, reduce throat irritation and improve the flavor of said formulations. The invention further provides vaporizable formulations comprising defined concentrations of the diluent(s) and cannabinoids. Use of the vaporizable formulations is provided as well.

### RELATED APPLICATION

This application is a Continuation-in-Part that claims priority to PCT International Application No. PCT/IL2020/050416, filed Apr. 6, 2020; which claims priority to foreign Israel Application No. 266151, filed Apr. 18, 2019; each of which are incorporated by reference.

### FIELD OF THE INVENTION

The present invention relates to stock compositions comprising terpenes, vaporizable formulations comprising cannabinoids and said stock compositions and uses thereof.

### BACKGROUND OF THE INVENTION

Cannabis is known as a drug and for recreational purpose, for thousands of years. There was a renewed interest in its use in the last decades, when it was found suitable for the treatment of many of diseases. The traditional ways of administration are oral and inhalation (smoking or vaping).

Administration of cannabis extracts or oils by vaping is done by the use of ceramic cartridges filled with formulations. The cannabis extract is very thick (viscous liquid a semi-solid), therefore a diluent is typically added. In principle, the technology of ceramic cartridges permit use of thick material, but in this case non consistent dose is obtained.

In order to solve viscosity problem, diluents are added to extracts or oil. Typical diluents include propylene glycol, vegetable glycerin, PEG, coconut oil, medium chain triglycerides, ethanol and terpenes. Terpenes are commonly used with cannabis extract composition for several reasons such as safety, their presence in cannabis extract and possible pharmacologic effect. US 2016/0151328 describes compositions comprising a combination of triacetin, dipropylene glycol, iso-phytol, and/or phytol and at least two terpenes. Sesquiterpenes and diterpenes are present at low concentration in cannabis extracts, typically much less than 1% of each individual terpene. However, terpenes are not inert components and in many times negatively affect the flavor and cause side effects.

WO 2018/160827 describes composition comprising a purified cannabinoid and a purified terpene. WO 2018/173049 describes vaporizable compositions comprising cannabinol and optionally sleep inducing terpenes.

Cannabinoids have many potential medical uses. Compositions comprising cannabinoids were approved in some countries for treatment of several medical indications such as intractable childhood epilepsy, chronic pain, loss of appetite etc. It was also shown that administration of CBD together with tobacco smoking in fact reduced the smoking frequency (Morgan et al., Addictive Behaviors 38 (2013) 2433-2436).

In each particular case, a specifically designed diluent or blend of diluents is usually formulated. There is an unmet need for safe diluents that may be used with a plurality of extracts.

### SUMMARY OF THE INVENTION

It has been surprisingly found according to the present invention that two terpenes: natural bisabolol and phytol when used as a combination in vaporizable compositions comprising cannabinoids provided several beneficial effects. First of all, when the concentrations of these compounds were correctly selected, a very consistent dose administration of the cannabinoids was achieved. It is a well-known fact that many cannabinoid containing compositions for vaporization, in particular extracts, distillates and oil, have poor dose reproducibility. Typically, the dose changes upon emptying of a cartridge. Moreover, it is impossible to anticipate how the dose changes, whether it increases or decreases. This, of course, negatively affects the ability to provide a consistent and reproducible treatment. The problem was effectively solved by the formulations of the present invention. An additional obstacle that has to be overcome when handling compositions comprising high concentration of CBD is its crystallization. In addition, it was found that the combination of bisabolol and phytol prevented crystallization of CBD, what allowed increasing the content of CBD in the cartridge. The combination also provided good flavor and did not harm the smoking experience, which was close to that of smoking cannabis plant. This is very important for a compliance. Moreover, the combination prevented or diminished irritation which is typical to extracts, distillates or oil vaping. It was further found that bisabolol may be used as a sole diluent to stabilize CBD in vaporizable formulations and prevents its crystallization. It was further found that additional diluents having sufficiently high boiling point may be efficiently used in formulations comprising CBD and nicotine and that such formulations are useful in treating nicotine and/or tobacco smoking addiction and lead to reduction of smoked cigarettes. It was found the compositions of the present invention comprising nicotine in combination with a cannabinoid selected from CBD and mixture of CBD and CBDA provide highly reproducible and constant dose of the nicotine and the cannabinoid. This allows very efficient treatment of nicotine/tobacco smoking addiction. According to one aspect, the present invention provides a stock composition comprising a sesquiterpene and a diterpene, wherein the cumulative concentration of the sesquiterpene and diterpene is from 70 wt % to 100 wt %, and wherein the weight ratio of the sesquiterpene to diterpene is from 1:10 to 10:1. According to some embodiments, the sesquiterpene is bisabolol such as a natural bisabolol and the diterpene is phytol such as a natural phytol. According to some embodiments, the cumulative content of the bisabolol and phytol in the composition is from 90 wt % to 100 wt %, and the weight ratio between bisabolol and phytol is from 1:5 to 5:1. According to some embodiments, the present invention provides a composition consisting essentially of bisabolol and phytol, wherein the weight ratio between bisabolol and phytol is from 1:5 to 5:1. According to some embodiments, the stock composition is devoid of cannabinoids. According to some embodiments, the stock composition consists of said terpenes.

According to another aspect, the present invention provides a vaporizable formulation comprising from 5 to 60 wt % of the stock composition of the present invention and at least one cannabinoid, wherein the stock composition comprises a sesquiterpene and a diterpene, wherein the cumulative concentration of the sesquiterpene and diterpene is from 70 wt % to 100 wt %, and wherein the weight ratio of the sesquiterpene to diterpene is from 1:10 to 10:1. According to some embodiments, the cumulative content of sesquiterpene and diterpene is at least 10 wt % of the formulation.

According to the teaching of the present invention, any cannabinoid may be used. According to some embodiments, the cannabinoid is selected from the group consisting of cannabidiol (CBD), cannabidiolic acid (CBDA), tetrahydrocannabinol (THC), THC acetate, tetrahydrocannabinolic acid (THCA), cannabigerol (CBG), cannabigerolic acid (CBGA), salts thereof, derivatives thereof and combinations of the cannabinoids. According to some embodiments, the vaporizable formulation comprises THC and from 5 to 50 wt % of the stock composition. According to other embodiments, the vaporizable formulation comprises CBD and from 15 to 60 wt % of the stock composition. According to one embodiment, the formulation comprises CBD and THC, and from 10 to 35 wt % of the stock composition. According to another embodiment, the molar ratio of THC to CBD is from 5:1 to 1:5. According to certain embodiments, the formulation comprises CBD and CBDA, and from 10 to 35 wt % of the stock composition. According to another embodiment, the molar ratio of CBD and CBDA is from 5:1 to 1:5. According to some embodiments, cumulative concentration of the cannabinoid(s) in the vaporizable formulation is from 45 to 85 wt %.

According to a further aspect, the present invention provides a vaporizable formulation comprising from 3 wt % to 35 wt % of a sesquiterpene and from 3 wt % to 35 wt % of a diterpene and at least one cannabinoid. According to some embodiments, the sesquiterpene is bisabolol such as a natural bisabolol and the diterpene is phytol such as a natural phytol. According to the teaching of the present invention, any cannabinoid may be used. According to some embodiments, the cannabinoid is selected from the group consisting of cannabidiol (CBD), cannabidiolic acid (CBDA), tetrahydrocannabinol (THC), THC acetate, tetrahydrocannabinolic acid (THCA), cannabigerol (CBG), cannabigerolic acid (CBGA), salts thereof, derivatives thereof and combinations of the cannabinoids.

According to one embodiment, the vaporizable formulation comprises from 60 to 90 wt % THC, from 3 wt % to 12 wt % of phytol and from 3 wt % to 12 wt % of bisabolol. According to another embodiment, the vaporizable formulation comprises from 40 to 80 wt % CBD, from 3 wt % to 27 wt % of phytol and from 3 wt % to 27 wt % of bisabolol. According to yet another embodiment, the vaporizable formulation comprises from 30 to 55 wt % of CBD, from 30 to 55 wt % of THC, from 3 wt % to 27 wt % of phytol and from 3 wt % to 27 wt % of bisabolol. According to a further embodiment, the vaporizable formulation comprises from 30 to 60 wt % of CBD, from 10 to 55 wt % of CBDA, from 3 wt % to 27 wt % of phytol and from 3 wt % to 27 wt % of bisabolol. According to some embodiments, the weight ratio of sesquiterpene to diterpene is from 1:5 to 5:1 or from 1:3 to 3:1. According to certain embodiments, the bisabolol is α-(−)-bisabolol and the phytol is natural phytol. According to some embodiments, the cumulative content of sesquiterpene and diterpene is at least 10 wt % of the formulation.

As mentioned earlier, CBD in high concentration tends to crystallize spontaneously. It was previously shown that crystals form in CBD oil containing between 60 and 95% CBD. It is shown according to the teaching of the present invention that an amount of 30% bisabolol is required to obtain a crystal resistant solution comprising up to 65 wt % CBD (originated from CBD oil comprising 92 wt % of CBD), which prevented CBD crystallization even when crystallization seeds are added. It was surprisingly found that even amount of 5% to 25% bisabolol may be sufficient to form a stable solution of CBD.

According to yet another aspect, the present invention provides a vaporizable formulation, comprising from 3 to 45 wt % of bisabolol and at least one cannabinoid. According to one embodiment, the vaporizable formulation comprises from 10 to 30 wt % bisabolol. According to some embodiments, the bisabolol is α-(−)-bisabolol. According to the teaching of the present invention, any cannabinoid may be used. According to some embodiments, the cannabinoid is selected from the group consisting of cannabidiol (CBD), cannabidiolic acid (CBDA), tetrahydrocannabinol (THC), THC acetate, tetrahydrocannabinolic acid (THCA), cannabigerol (CBG), cannabigerolic acid (CBGA), salts thereof, derivatives thereof and combinations of the cannabinoids. According to some embodiments, the vaporizable formulation, comprises from 60 to 87 wt % of CBD and from 5 to 25% bisabolol.

According to some embodiments, the vaporizable formulation further comprises nicotine.

According to any one of the above aspects and embodiments, the vaporizable formulation according to the teaching of the present invention upon vaporization provides a constant dose of the cannabinoid. According to some embodiments the deviation between the doses of a cannabinoid upon multiple vaporizations is no more than 10%.

According to another aspect, the present invention provides a vaporizable formulation comprising CBD, from 0.5 to 10 wt % of nicotine and at least one diluent having sufficiently high boiling point. According to some embodiments, the formulation comprises from 50 to 90 wt % of the diluent, and from 5 to 25 wt % of CBD. According to some embodiments, the formulation comprises from 50 to 90 wt % of CBD of the diluent, and from 5 to 35 wt % of the diluent. According to some embodiments, some of the CBD is replaced by CBDA.

According to one aspect, the vaporizable formulation of the present invention is for use in treating a disease or a disorder treatable with a cannabinoid.

According to another aspect, the present invention provides a method of treating a disease of a conditions treatable with a cannabinoid in a subject in need thereof, comprising administering by vaporization the vaporizable formulation of the present invention.

According to some embodiments, the vaporizable formulation of the present invention comprising nicotine is for treatment of nicotine tobacco addiction. According to some embodiments, the vaporizable formulation of the present invention comprising nicotine is for treatment of tobacco smoking addiction.

According to yet another aspect, the present invention provides a kit comprising (i) the stock composition of the present invention comprising a sesquiterpene and a diterpene, wherein the cumulative concentration of the sesquiterpene and diterpene is from 70 wt % to 100 wt %, and wherein the weight ratio of the sesquiterpene to diterpene is from 1:10 to 10:1, (ii) a composition comprising at least one cannabinoid and (iii) instructions to use said compositions for preparation of a vaporizable formulation.

### BRIEF DESCRIPTION OF DRAWINGS

FIG. 1 shows the change of the dose upon emptying the cartridge filled with different compositions FIG. 1A (combination 1), FIG. 1B (combination 2), FIG. 1C (combination 3), FIG. 1D (combination 4), and FIG. 1E (combination 5).

FIG. 2 shows the chromatograms of formulations comprising the THC-rich distilled extract in cartridge (FIG. 2A) and in the vaporized aerosol (FIG. 2B).

### DETAILED DESCRIPTION OF THE INVENTION

According to one aspect, the present invention provides a composition comprising a sesquiterpene and a diterpene, wherein the cumulative concentration of the sesquiterpene and diterpene is from 70 wt % to 100 wt %, and wherein the weight ratio of the sesquiterpene to diterpene is from 1:10 to 10:1.

The term “terpene” or “terpenes” are used herein interchangeably and refer to terpenes naturally present in cannabis plant. The term terpene or terpenes also includes terpenoids.

According to some embodiments, the terpenes are selected from sesquiterpene, diterpene and a combination thereof. Non-limiting examples of sesquiterpenes are caryophyllene, caryophyllene oxide, humulene, alpha-humulene, alpha-bisabolene; beta bisabolene; Santalol; selinene; nerolidol, bisabolol; alpha cedrene, beta-cedrene, beta-eudesmol, eudesm-7(11)-en-4-ol, Selina-3.7(11)-diene, guaiol, valencene, alpha-guaiene, beta-guaiene, delta-guaiene, guaiene, farnesene, alpha-farne sene, beta-farnesene, elemene, alpha-elemene, beta-elemene, gamma-elemene, delta-elemene, germacrene, germacrene A, germacrene B. germacrene C, germacrene D, and germacrene E. According to some embodiments, the sesquiterpene is humulene or B-carrophylene. According to certain embodiments, the sesquiterpene is a combination of bisabolol and humulene and/or B-carrophylene. According to one embodiment, the sesquiterpene is selected from valenciene, humulene, cadinene, caryophyllene, caryophyllene oxide. According to another embodiment, the sesquiterpene is a combination of natural bisabolol, valenciene, humulene, cadinene, caryophyllene, and caryophyllene oxide. Non limiting examples of diterpenes are oridonin, phytol, and isophytol. According to some embodiment the phytol is cis-phytol, trans-phytol or a racemate mixture of the two.

The terms “composition” and “stock composition” are used herein interchangeably and refer to a composition comprising mainly terpenes such as a sesquiterpene and a diterpene and is used for preparation of formulations, such as vaporizable formulations comprising cannabinoids. According to some embodiments, the stock composition is devoid of a traceable amount of cannabinoids. Thus, according to some embodiments, the present invention provides a stock composition comprising a sesquiterpene and a diterpene, wherein the cumulative concentration of the sesquiterpene and diterpene is from 70 wt % to 100 wt %, and wherein the weight ratio of the sesquiterpene to diterpene is from 1:10 to 10:1.

According to one embodiment, the sesquiterpene is bisabolol. According to another embodiment, the diterpene is phytol. According to some embodiments, the present invention provides a stock composition comprising a sesquiterpene and a phytol, wherein the cumulative concentration of the sesquiterpene and phytol is from 70 wt % to 100 wt %, and wherein the weight ratio of the sesquiterpene to phytol is from 1:10 to 10:1.

According to one embodiment, the sesquiterpene is bisabolol and the diterpene is phytol. Thus, according to one embodiment, the present invention provides a stock composition comprising a bisabolol and a phytol, wherein the cumulative concentration of the bisabolol and phytol is from 70 wt % to 100 wt %, and wherein the weight ratio of the sesquiterpene to diterpene is from 1:10 to 10:1.

According to some embodiments, the bisabolol is a natural bisabolol, i.e. α-(−)-bisabolol. According to another embodiment, the bisabolol is a racemate of α-(−)-bisabolol and a-(+)-bisabolol.

According to some embodiments, the phytol is a natural phytol, i.e. (2E,7R,11R)-3,7,11,15-tetramethyl-2-hexadecen-1-ol. According to one embodiment, the phytol is a mixture of isomers of 3,7,11,15-tetramethyl-2-hexadecen-1-ol. According to another embodiment, the bisabolol is a natural bisabolol, i.e. α-(−)-bisabolol and the phytol is a mixture of isomers of 3,7,11,15-tetramethyl-2-hexadecen-1-ol. According to yet another embodiment, the bisabolol is a natural bisabolol, i.e. α-(−)-bisabolol and the phytol is a natural phytol.

According to some embodiments, the cumulative content of the sesquiterpene and diterpene in the stock composition is from 75 to 95 wt % or from 80 to 90 wt %. According to one embodiment, the cumulative content of the sesquiterpene and diterpene in the composition is from 85 to 100 wt % or from 90 to 100 wt %. According to another embodiment, the cumulative content of the sesquiterpene and diterpene in the stock composition is from 92 to 100 wt %, from 95 to 100 wt %, or from 97 to 100 wt %. According to another embodiment, the cumulative content of the sesquiterpene and diterpene in the stock composition is from 80 to 95 wt % from 85 to 95 wt % or from 85 to 98 wt %.

According to some embodiments, the cumulative content of the sesquiterpene and phytol in the composition is from 75 to 95 wt % or from 80 to 90 wt %. According to one embodiment, the cumulative content of the sesquiterpene and phytol in the stock composition is from 85 to 100 wt % or from 90 to 100 wt %. According to another embodiment, the cumulative content of the sesquiterpene and phytol in the stock composition is from 92 to 100 wt %, from 95 to 100 wt %, or from 97 to 100 wt %. According to another embodiment, the cumulative content of the sesquiterpene and phytol in the stock composition is from 80 to 95 wt % from 85 to 95 wt % or from 85 to 98 wt %.

According to some embodiments, the cumulative content of the bisabolol and phytol in the stock composition is from 75 to 95 wt % or from 80 to 90 wt %. According to one embodiment, the cumulative content of the bisabolol and phytol in the composition is from 85 to 100 wt % or from 90 to 100 wt %. According to another embodiment, the cumulative content of the bisabolol and phytol in the stock composition is from 92 to 100 wt %, from 95 to 100 wt %, or from 97 to 100 wt %. According to another embodiment, the cumulative content of the bisabolol and phytol in the stock composition is from 80 to 95 wt % from 85 to 95 wt % or from 85 to 98 wt %.

According to some embodiments, the molar ratio of bisabolol, e.g. α-(−)-bisabolol and phytol, e.g. natural phytol is from 1:10 to 10:1, from 1:9 to 9:1, from 1:8 to 8:1, from 1:7 to 7:1 or from 1:6 to 6:1. According to one embodiment, the molar ratio of bisabolol, e.g. α-(−)-bisabolol and natural phytol is from 1:5 to 5:1, from 1:4 to 4:1, from 1:3 to 3:1 or from 1:2 to 2:1. According to some embodiments, the molar ratio of bisabolol, e.g. α-(−)-bisabolol and natural phytol is from 1:2 to 2:1, or about 1:1. According to some embodiments, the molar ratio of bisabolol, e.g. α-(−)-bisabolol and natural phytol is selected from 1:2, 2:1 and 1:1.

According to some embodiments, the cumulative content of the bisabolol and phytol is from 90 wt % to 100 wt %, and the weight ratio between α-(−)-bisabolol and phytol e.g. natural phytol is from 1:5 to 5:1, or from 4:1 to 1:4, or from 1:1 to 1:4. According to another embodiment, the cumulative content of the natural bisabolol and phytol in the composition is from 92 to 100 wt %, from 95 to 100 wt %, from 95 to 98 wt % or from 97 to 100 wt %. According to some embodiments, the cumulative content of the natural bisabolol and phytol in the composition is about 90, 91, 92, 93, 94, 95, 96, 97, 98, 99 or about 99 wt %.

The term “comprises” has also the meaning of consisting and may be substituted by it. Thus, in one embodiment, the stock composition consists essentially of bisabolol and phytol, wherein the weight ratio between bisabolol and phytol is from 1:5 to 5:1. According to some embodiments, the stock composition consists essentially of bisabolol and phytol, wherein the weight ratio between bisabolol and phytol is from 1:2 to 2:1. According to some embodiments, the stock composition consists essentially of bisabolol and phytol, wherein the weight ratio between bisabolol and phytol is from 1:3 to 3:1. According to some embodiments, the stock composition consists essentially of bisabolol and phytol, wherein the weight ratio between bisabolol and phytol is from 1:4 to 4:1. According to yet another embodiment, the stock composition consist of a bisabolol and a phytol. According to a further embodiment, the stock composition consist of a bisabolol and a phytol, and optionally one or more pharmaceutically acceptable excipients.

According to any one of the above embodiments, the composition further comprises an antioxidant. According to one embodiment, the antioxidant is selected from citric acid, ascorbyl palmitate and a combination thereof. According to some embodiments, the antioxidant is selected from citric acid, ascorbyl palmitate, malic acid, tartaric acid oxalic acid, succinic acid and ascorbyl stearate, and any combination thereof. According to some embodiments, the formulation comprises from 0.001 to 1 wt % of the antioxidants, from 0.05 to 0.9, from 0.1 to 0.6 from 0.2 to 0.5 wt % of the antioxidant. According to some embodiments, the formulation comprises from 0.005 to 0.5 wt % of the antioxidant, from 0.01 to 0.4 wt %, from 0.03 to 0.35 wt %, from 0.05 to 0.3 wt % of the antioxidant.

According to some embodiments, the composition is for use as a diluent in a vaporizable formulation. The term “diluent” as used herein refers to a compound or a mixture of compounds that reduces the viscosity of cannabis extract, oil, distillate or isolate, and/or prevents cannabinoid crystallization. According to some embodiments, the stock composition of the present invention reduces the viscosity of cannabis extract, distillate, oil, or isolate. According to other embodiments, the stock composition of the present invention prevents or inhibits crystallization of cannabinoids, e.g. crystallization of CBD. According to one embodiment, the stock composition of the present invention is configured for vaporization.

According to some embodiments, the composition has a synergic effect on reducing throat irritation and on the flavor of the vaporizable formulation in which the composition is used. As use herein, the term “synergistic effect” means that the combination of the components of the composition exhibits a greater effect or activity than the additive effect or activity provided when each component of the combination is applied alone.

According to some embodiments, the stock composition of the present invention comprising a sesquiterpene and a diterpene is devoid of any cannabinoids. Thus, according to some embodiments, the present invention provides a stock composition comprising a sesquiterpene and a diterpene, wherein the cumulative concentration of the sesquiterpene and diterpene is from 70 wt % to 100 wt %, wherein the weight ratio of the sesquiterpene to diterpene is from 1:10 to 10:1, and wherein the stock composition is devoid of a cannabinoid. All above embodiments referring to stock compositions comprising a sesquiterpene and a diterpene apply herein as well. According to some embodiments, the cumulative content of the sesquiterpene and diterpene is from 90 wt % to 100 wt %. According to some embodiments, the composition consists of the sesquiterpene and diterpene. According to certain embodiments, the weight ratio between sesquiterpene and diterpene is from 1:5 to 5:1. According to one embodiment, the sesquiterpene is a bisabolol. According to another embodiment, the diterpene is a phytol. According to yet another embodiment, the sesquiterpene is bisabolol and the diterpene is phytol. According to one embodiment, bisabolol is α-(−)-bisabolol. According to another embodiment, the diterpene is natural phytol. Thus, according to some embodiments, the present invention provides a composition consisting of bisabolol and phytol, wherein the weight ratio between bisabolol and phytol is from 1:5 to 5:1.

According to another aspect, the present invention provides a vaporizable formulation comprising from 5 to 70 wt % or from 5 to 60 wt % of the stock composition according to any one of the above or below embodiments and aspects and at least one cannabinoid. Thus, in some embodiments, the present invention provides a vaporizable formulation comprising from 5 to 60 wt % of the stock composition of the present invention comprising a sesquiterpene and a diterpene, and at least one cannabinoid, wherein the cumulative concentration of the sesquiterpene and diterpene in the stock composition is from 70 wt % to 100 wt %, and wherein the weight ratio of the sesquiterpene to diterpene in the stock composition is from 1:10 to 10:1. According to one embodiment, the sesquiterpene is bisabolol such as α-(−)-bisabolol. According to another embodiments, the diterpene is phytol such as natural phytol. According to yet another embodiment, the sesquiterpene is bisabolol such as α-(−)-bisabolol and the diterpene is phytol such as natural phytol. In some embodiments, the present invention provides a vaporizable formulation comprising from 5 to 60 wt % of the stock composition of the present invention comprising a sesquiterpene and a phytol, and at least one cannabinoid, wherein the cumulative concentration of the sesquiterpene and phytol in the stock composition is from 70 wt % to 100 wt %, and wherein the weight ratio of the sesquiterpene to diterpene in the stock composition is from 1:10 to 10:1.

According to the teaching of the present invention, the cumulative content of sesquiterpene and phytol is at least 10 wt % of the vaporizable formulation. Thus, according to one embodiment, the present invention provides a vaporizable formulation comprising from 5 to 70 wt % or from 5 to 60 wt % of the stock composition according to any one of the above or below embodiments and aspects and at least one cannabinoid, wherein the cumulative content of sesquiterpene and diterpenes is at least 10 wt % of the vaporizable formulation. According to some embodiments, the cumulative content of sesquiterpene and phytol is at least 10 wt % of the vaporizable formulation. According to another embodiment, the cumulative content of a bisabolol and a phytol is at least 10 wt % of the vaporizable formulation. According to some embodiments, the cumulative content of sesquiterpene and diterpenes such as bisabolol and phytol is at least 15 wt % of the vaporizable formulation. According to some embodiments, the cumulative content of sesquiterpene and diterpenes such as bisabolol and phytol is at least 20 wt % of the vaporizable formulation. According to some embodiments, the cumulative content of sesquiterpene and diterpenes such as bisabolol and phytol is at least 25 wt % of the vaporizable formulation. According to some embodiments, the cumulative content of sesquiterpene and diterpenes such as bisabolol and phytol is at least 30 wt % of the vaporizable formulation. According to some embodiments, the cumulative content of sesquiterpene and diterpenes such as bisabolol and phytol is at least 35 or 40 wt % of the vaporizable formulation. According to some embodiments, the cumulative content of bisabolol and phytol is at least 10 wt %, at least 15 wt %, at least 20 wt %, at least 25 wt % or 30 wt % of the vaporizable formulation.

The term “vaporizable formulation” as used herein refers to a formulation that, when used in a suitable device, converts to a vapor or an aerosol that can be inhaled. In one embodiment, the vaporizable composition produces inhalable vapor upon heating e.g. using a vaporizer such as electronic cigarettes. According to some embodiments, the vaporization occurs in the temperature range of between about 120 to about 240° C. According to some embodiments, the composition of the present invention is vaporizable at the temperature range of between about 120° C. to about 250° C. According to another embodiment, the composition is vaporizable in the temperature range of between about 130° C. to about 230° C., about 140° C. to about 220° C., or about 150° C. to about 210° C. According to one embodiment, the composition is vaporizable in the temperature range of between about 160° C. to about 230° C. or about 170 to about 220° C.

According to some embodiments, the vaporizable formulation comprises from 5 to 60 wt %, 10 to 60 wt %, from 15 to 55 wt %, from 20 to 50 wt %, from 5 to 45 wt % or from 30 to 40 wt % of the stock composition of the present invention. According to some embodiments, the vaporizable formulation comprises from 10 to 45 wt %, from 15 to 40 wt %, from 20 to 35 wt % or from 25 to 30 wt % of the stock composition. According to some embodiments, the formulation comprises from 10 to 30 wt %, or from 15 to 25 wt % or from 10 to 20 wt % of the stock composition. According to other embodiments, the vaporizable formulation comprises from 15 to 45 wt %, from 20 to 40 wt % or from 25 to 35 wt % of the stock composition. According to other embodiments, the vaporizable formulation comprises from 20 to 60 wt %, from 25 to 55 wt %, from 30 to 50 wt %, from 35 to 45 wt %, from 20 to 40 wt %, from 30 to 50 wt % or from 40 to 60 wt % of the stock composition.

According to some embodiments, the cumulative content of sesquiterpene and diterpene is from 10 to 60 wt % of the vaporizable formulation. According to some embodiments, the cumulative content of sesquiterpene and diterpene is from 15 to 55 wt % of the vaporizable formulation. According to some embodiments, the cumulative content of sesquiterpene and diterpene is from 20 to 50 wt % of the vaporizable formulation. According to some embodiments, the cumulative content of sesquiterpene and diterpene is from 10 to 40 wt % of the vaporizable formulation. According to some embodiments, the cumulative content of sesquiterpene and diterpene is from 25 to 45 wt %, from 30 to 40 wt % of the vaporizable formulation. According to some embodiments, the cumulative content of sesquiterpene and diterpene is from 15 to 55 wt % of the vaporizable formulation. According to some embodiments, the cumulative content of sesquiterpene and diterpene is from 10 to 30 wt %, or from 20 to 60 wt %, or from 15 to 45 wt % or from 30 to 60 wt % of the vaporizable formulation. According to some embodiments, the cumulative content of sesquiterpene and diterpene is from 10 to 40 wt %. According to other embodiments, the cumulative content of sesquiterpene and diterpene is from 20 to 50 wt %. According to some embodiments, the cumulative content of sesquiterpene and phytol is from 10 to 40 wt %. According to some embodiments, the cumulative content of sesquiterpene and phytol is from 20 to 50 wt %. According to some embodiments, the cumulative content of sesquiterpene and phytol is from 10 to 60 wt %, from 15 to 55 wt %, from 20 to 50 wt %, from 10 to 40 wt %, from 25 to 45 wt %, from 30 to 40 wt %, from 10 to 30 wt %, or from 20 to 60 wt %, or from 15 to 45 wt % or from 30 to 60 wt % of the vaporizable formulation. According to some embodiments, the cumulative content of sesquiterpene and phytol is from 15 to 40 wt % or from 15 to 35 wt % of the vaporizable formulation. According to some embodiments, the cumulative content of bisabolol and phytol is from 10 to 60 wt %, from 15 to 55 wt %, from 20 to 50 wt %, from 10 to 40 wt %, from 25 to 45 wt %, from 30 to 40 wt %, from 10 to 30 wt %, or from 20 to 60 wt %, or from 15 to 45 wt % or from 30 to 60 wt % of the vaporizable formulation. According to some embodiments, the cumulative content of bisabolol and phytol is from 15 to 40 wt % or from 15 to 35 wt % of the vaporizable formulation.

According to any one of the above embodiments, the sesquiterpene is bisabolol and the diterpene is a phytol. According to some embodiments, the cumulative content of bisabolol and phytol is from 10 to 60 wt % of the vaporizable formulation. In alternative embodiments, the vaporizable formulation comprises from 10 to 60 wt %, from 15 to 55 wt %, from 20 to 50 wt %, from 25 to 45 wt %, from 30 to 40 wt %, from 10 to 30 wt %, from 30 to 60 wt %, from 20 to 40 wt % of sesquiterpene and diterpene, wherein the weight ratio between sesquiterpene and diterpene is from 1:10 to 10:1 or from 1:5 to 5:1. In some embodiments, the vaporizable formulation comprises from 10 to 60 wt %, from 15 to 55 wt %, from 20 to 50 wt %, from 25 to 45 wt %, from 30 to 40 wt %, from 10 to 30 wt %, from 30 to 60 wt %, from 20 to 40 wt % of sesquiterpene and phytol, wherein the weight ratio between sesquiterpene and phytol is from 1:5 to 5:1. In another embodiment, the vaporizable formulation comprises from 10 to 60 wt %, from 15 to 55 wt %, from 20 to 50 wt %, from 25 to 45 wt %, from 30 to 40 wt %, from 10 to 30 wt %, from 30 to 60 wt %, from 20 to 40 wt % of bisabolol and phytol, wherein the weight ratio between bisabolol and phytol is from 1:5 to 5:1.

The term “cannabinoid” as used herein refers to a cannabinoid naturally present in cannabis plant and acts on cannabinoid receptors, or derivative of said cannabinoid. It is understood that cannabinoids from any source may be used. The cannabinoid may be purified cannabinoid, such as an isolate, cannabinoid present in cannabis oil, extract or cannabis plant, a distillate of cannabis oil or of extract or a synthetic cannabinoid. Cannabinoids that are derived from cannabis oil may further comprise terpenes naturally present in cannabis oil. For vaporizable formulations of the present invention, a distillate (e.g. of cannabis oil or of extract) is preferred.

According to any one of the above embodiments, the cannabinoid is selected from the group consisting of cannabidiol (CBD), cannabidiolic acid (CBDA), tetrahydrocannabinol (THC), tetrahydrocannabinolic acid (THCA), THC acetate, cannabigerol (CBG), cannabigerolic acid (CBGA), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), tetrahydrocannabivarinic acid (THCVA), cannabidivarin (CBDV), cannabichromevarin (CBCV), cannabichromevarinic acid (CBCVA), cannabigerovarin (CBGV), cannabichromenic acid (CBCA), cannabidivarinic acid (CBDVA) cannabigerovarinic acid (CBGVA) and cannabigerol monomethyl ether (CBGM), salts thereof, derivatives thereof and mixtures of cannabinoids.

According to some embodiments, the vaporizable formulation comprises from 50 to 90 wt % of the cannabinoid. According to some embodiments, the vaporizable formulation comprises from 55 to 85 wt %, from 60 to 80 wt %, or from 65 to 75 wt % of the cannabinoid. According to some embodiments, the vaporizable formulation comprises from 10 to 70 wt %, from 10 to 30 wt %, from 30 to 50 wt % or from 50 to 80 wt % of the cannabinoid.

The term “cannabis” as used herein, refers to plants of the genus Cannabis, including but not limited to Cannabis sativa, Cannabis indica, and Cannabis ruderalis.

The term “extract” as used herein refers to a liquid substance obtained through extraction from a given substance. In particular, the term “extract” refers to a liquid or semi-solid or resinous substance obtained through extraction from cannabis plant. In some embodiments, the term refers to a mixture of liquid or semi-solid, resinous substances obtained through extraction from two or more different cannabis species. In some embodiments, the term refers also to a compound purified from the extract. According to some embodiments, the term “extract” has the meaning of a mixture or combination of two or more extracts. In some embodiments, the term “extract” and “cannabis oil” may be used interchangeably. According to some embodiments, the cannabis oil or distillate comprises from 70 to 95 wt % of CBD. According to other embodiments, the cannabis oil comprises from 75 to 90 wt % of CBD, from 80 to 95 wt %, or from 85 to 95 wt % of CBD. According to some embodiments, the cannabis oil comprises about 85, 86, 87, 88, 89, 90, 91, 92 or 93 wt % of CBD. According to other embodiments, the cannabis oil or distillate comprises from 70 to 95 wt % of THC. According to other embodiments, the cannabis oil or distillate comprises from 75 to 90 wt % of THC, from 80 to 95 wt %, from 85 to 95 wt % of THC. According to some embodiments, the cannabis oil or distillate comprises about 85, 86, 87, 88, 89, 90, 91, 92 or 93 wt % of THC.

The term “isolate” with respect to a cannabinoid refers to a purified cannabinoid, comprising about 98-100 wt % of said cannabinoid.

According to some embodiments, the cannabinoid is originated from a distillate of cannabis extract. The term “distillate” refers to a solution that has been separated by any known means of evaporation or distillation. In the methods of the invention, the cannabis extract or oil may be further distilled to obtain a cannabinoid distillate.

According to some embodiments, cannabis distillate comprises from 70 to 95 wt % of CBD. According to other embodiments, the cannabis distillate comprises from 75 to 90 wt % of CBD, from 80 to 95 wt %, from 85 to 95 wt % of CBD. According to some embodiments, the cannabis distillate comprises about 85, 86, 87, 88, 89, 90, 91, 92 or 93 wt % of CBD.

According to some embodiments, the cannabinoid is THC. According to some embodiments, the formulation comprises THC and from 5 to 50 wt % of the stock composition. According to another embodiment, the formulation comprises from 5 to 25 wt % of the composition of the present invention. According to some embodiments, the formulation comprises from 10 to 20 wt % of the stock composition. According to other embodiments, the formulation comprises from 10 to 40 wt %, from 15 to 35 wt % or from 20 to 30 wt % of the stock composition. According to some such embodiments, the formulation comprises from 50 to 90 wt % of THC. According to one embodiment, the formulation comprises from 55 to 85 wt %, from 60 to 80 wt % or from 65 to 75 wt % of THC. According to one embodiment, the formulation comprises from 70 to 95 wt % THC-rich extract (about 85% THC), from 6 to 9 wt % bisabolol and from 6 to 9 wt % phytol. According to some embodiments, the cumulative content of sesquiterpene and diterpenes such as bisabolol and phytol is at least 10 wt % of the vaporizable formulation. According to some embodiments, the cumulative content of sesquiterpene and diterpenes such as bisabolol and phytol is at least 15 wt %, at least 20 wt %, or at least 25 wt % of the vaporizable formulation. According to another embodiment, the cumulative content of bisabolol and phytol is at least 10 wt % of the formulation. According to yet another embodiment, the cumulative content of bisabolol and phytol is at least 15 wt % of the formulation. According to some embodiments, the cumulative content of bisabolol and phytol is at least 15 wt % of the formulation.

According to another embodiment, the cannabinoid is CBD. According to some embodiments, the formulation comprises CBD and from 15 to 50 wt % of the stock composition. According to some embodiments, the formulation comprises from 20 to 35 wt % or from 25 to 30 wt % of the stock composition. According to some such embodiments, the formulation comprises from 30 to 85 wt % of CBD. According to other embodiments, the formulation comprises from 55 to 80 wt %, from 60 to 75 wt % or from 65 to 70 wt % of CBD. According to some embodiments, the formulation comprises from 70 to 85 wt % of CBD. According to some embodiments, the formulation comprises from 10 to 35 wt % of CBD. According to some embodiments, the cumulative content of sesquiterpene and diterpenes such as bisabolol and phytol is at least 15 wt % of the vaporizable formulation. According to some embodiments, the cumulative content of sesquiterpene and diterpenes such as bisabolol and phytol is at least 20 wt %, at least 25 wt %, or at least 30 wt % of the vaporizable formulation. According to another embodiment, the cumulative content of bisabolol and phytol is at least 15 wt % of the formulation.

According to some embodiments, the formulation comprises a combination of cannabinoids. According to certain embodiments, the combination is selected from a combination of CBD and THC; combination of CBD and CBN, combination of CBD and CBDA; combination of CBD and CBG. According to one embodiment, the vaporizable formulation comprises a combination of CBD and THC, and from 5 to 35 wt % of the stock composition. According to one embodiment, the vaporizable formulation comprises a combination of CBD and THC, and from 10 to 35 wt % of the stock composition. According to some embodiments, the formulation comprises from 15 to 30 wt %, or 20 to 25 wt % of the stock composition of the present invention. According to some embodiments, the molar ratio of THC to CBD is from 5:1 to 1:5. According to another embodiment, the molar ratio of THC to CBD is from 4:1 to 1:4 or from 2:1 to 1:2. According to some embodiments, the cumulative concentration of the THC and CBD is from 60 to about 85 wt %, or from 65 to 85 wt % of from 70 to 80 wt % of the formulation.

According to some embodiments, the vaporizable formulation comprises a combination of CBD and CBDA. According to some such embodiments, the formulation comprises a combination of CBD and CBDA and from 5 to 35 wt % of the stock composition of the present invention. According to some such embodiments, the formulation comprises a combination of CBD and CBDA and from 10 to 35 wt % of the stock composition. According to some embodiments, the formulation comprises from 15 to 30 wt %, or from 20 to 25 wt % of the stock composition of the present invention. According to other embodiments, the formulation comprises from 10 to 40 wt %, from 10 to 20 wt %, from 15 to 35 wt % or from 20 to 30 wt % of the stock composition. According to some embodiments, the molar ratio of CBD to CBDA is from 5:1 to 1:5. According to another embodiment, the molar ratio of CBD to CBDA is from 4:1 to 1:4, 3:1 to 1:3, from 2:1 to 1:2 or about 1:1. According to some embodiments, the cumulative concentration of the CBD and CBDA is from 60 to about 90 wt %, or from 65 to 85 wt % or from 70 to 80 wt % of the formulation. According to some embodiments, the cumulative content of sesquiterpene and diterpenes such as bisabolol and phytol is at least 10 wt % of the vaporizable formulation. According to some embodiments, the cumulative content of sesquiterpene and diterpenes such as bisabolol and phytol is at least 15 wt %, at least 20 wt %, or at least 25 wt % of the vaporizable formulation. According to another embodiment, the cumulative content of bisabolol and phytol is at least 10 wt % of the formulation. According to another embodiment, the cumulative content of bisabolol and phytol is at least 15 wt % of the formulation.

According to yet another embodiment, the formulation comprises a combination of CBD and CBN. According to some embodiments, the molar ration of CBD to CBN is from 5:1 to 1:5. According to another embodiment, the molar ratio of CBD to CBN is from 4:1 to 1:4, 3:1 to 1:3, from 2:1 to 1:2 or about 1:1. According to some embodiments, the molar ratio of CBD to CBN is from 20:1 to 1:1 or from 15:1 to 5:1.

According to any one of the above or below embodiments, the vaporizable formulation is devoid of crystals of cannabinoids. According to some embodiments, the vaporizable formulation comprising CBD is devoid of CBD crystals. According to some embodiments, the vaporizable formulation prevent crystallization of cannabinoids, such as CBD crystallization.

It is understood according to the teaching of the present invention that terpenes of the formulation of the present invention originate from the stock composition of the present invention. However, in certain embodiments, some of the sesquiterpene and/or some of the diterpenes originate from the cannabinoid oil, extract or distillate.

According to any one of the above embodiments, the vaporizable formulation further comprises nicotine. According to some embodiments, the nicotine is provided as a tobacco extract. Thus, according to one embodiment, the vaporizable formulation further comprises a tobacco extract. According to one embodiment, the vaporizable formulation comprises from 0.1 to 7 wt % nicotine, from 0.2 to 5 wt % nicotine, from 0.3 to 3 or from 0.5 to 2 wt % nicotine. The term “nicotine” refers to the basic form and to a salt of the nicotine such as benzoic acid salt, salicylic acid, sorbic acid, benzoic acid, lauric acid, levulinic acid and malic acid. According to some embodiments, the vaporizable formulation comprises from 10 to 40 wt % of the stock composition, from 40 to 75 wt % of CBD and from 0.5 to 3 wt % of nicotine. According to some embodiments, the vaporizable formulation comprises from 0.5 to 4 wt %, from 1 to 3 wt %, from 1.5 to 2.5 wt % of nicotine. According to some embodiments, the vaporizable formulation comprises from 1 to 3 wt % or about 2 wt % of nicotine. According to some embodiments, the vaporizable formulation comprises from 10 to 30 wt % of bisabolol, from 10 to 30 wt % of phytol, from 60 to 90 wt % of CBD and from 0.5 to 3 wt % of nicotine. According to other embodiments, the vaporizable formulation comprises from 10 to 20 wt % of bisabolol, from 10 to 20 wt % of phytol, from 60 to 80 wt % of CBD and from 1 to 3 wt % of nicotine. According to some embodiments, the vaporizable formulation comprises from 10 to 30 wt % of bisabolol, from 10 to 30 wt % of phytol, from 10 to 50 wt % of CBD and from 0.5 to 3 wt % of nicotine. According to other embodiments, the vaporizable formulation comprises from 10 to 30 wt % of bisabolol, from 10 to 30 wt % of phytol, from 30 to 60 wt % of CBD and from 1 to 3 wt % of nicotine. According to some embodiments, the vaporizable formulation comprises from 10 to 30 wt % of bisabolol, from 10 to 30 wt % of phytol, from 20 to 40 wt % of CBD and from 0.5 to 3 wt % of nicotine. According to some such embodiments, the formulation comprises from 10 to 60 wt %, from 15 to 55 wt %, from 20 to 50 wt %, from 25 to 45 wt %, from 30 to 40 wt %, from 35 to 60 wt %, or from 10 to 25 wt %, from 15 to 20 wt %, from 10 to 15 wt %, or from 45 to 55 wt % of CBD.

According to any one of the above embodiments, the formulation further comprises an antioxidant. According to one embodiment the antioxidant is selected from citric acid, ascorbyl palmitate and a combination thereof According to some embodiments, the antioxidant is selected from citric acid, ascorbyl palmitate, malic acid, tartaric acid oxalic acid, succinic acid and ascorbyl stearate, and any combination thereof. According to some embodiments, the formulation comprises from 0.001 to 1 wt % of the antioxidants, from 0.05 to 0.9, from 0.1 to 0.6 from 0.2 to 0.5 wt % of the antioxidant. According to some embodiments, the formulation comprises from 0.005 to 0.5 wt % of the antioxidant, from 0.01 to 0.4 wt %, from 0.03 to 0.35 wt %, from 0.05 to 0.3 wt % of the antioxidant.

According to any one of the above embodiments, the formulation further comprises an anti-irritant, such as menthol.

According to some embodiments, the present invention provides a vaporizable formulation comprising from 3 wt % to 35 wt % of a sesquiterpene and from 3 wt % to 35 wt % of a diterpene and at least one cannabinoid. According to one embodiment, the diterpene is phytol. According to another embodiment, the sesquiterpene is bisabolol. According to one embodiment, the diterpene is phytol and the sesquiterpene is bisabolol. According to some embodiments, the present invention provides a vaporizable formulation comprising from 3 wt % to 35 wt % of a sesquiterpene and from 3 wt % to 35 wt % of a phytol and at least one cannabinoid. According to one embodiment, the bisabolol is α-(−)-bisabolol. According to one embodiment, the phytol is natural phytol. According to one embodiment the cumulative content of sesquiterpene and phytol is at least 10 wt % of the formulation. According to one embodiment the cumulative content of sesquiterpene and phytol is at least 15 wt % of the formulation. According to one embodiment, the vaporizable formulation comprises from 5 to 30 wt %, from 5 to 20 wt %, from 5 to 15 wt %, from 10 to 25 wt %, from 15 to 20 wt % or from 10 to 20 wt % of sesquiterpene such as bisabolol. According to another embodiment, the vaporizable formulation comprises from 5 to 20 wt %, from 5 to 15 wt %, from 5 to 30 wt %, from 10 to 25 wt %, from 15 to 20 wt % or from 10 to 20 wt % of diterpene such as phytol. According to yet another embodiment, the vaporizable formulation comprises from 5 to 30 wt %, from 10 to 25 wt %, from 5 to 15 wt %, from 10 to 25 wt %, from 15 to 20 wt % of the sesquiterpene such as bisabolol and from 5 to 30 wt %, from 5 to 15 wt %, from 10 to 25 wt %, from 10 to 25 wt %, from 15 to 20 wt % of the diterpene such as phytol. According to one embodiment, the bisabolol is α-(−)-bisabolol. According to one embodiment, the cumulative content of sesquiterpene and phytol is at least 10 wt % of the formulation. According to another embodiment the cumulative content of sesquiterpene and phytol is at least 15 wt % of the formulation. According to one embodiment, the formulation further comprises an additional sesquiterpene selected from valenciene, humulene, cadinene, caryophyllene, caryophyllene oxide and any combination thereof. According to another embodiment, the formulation comprises a combination of natural bisabolol, valenciene, humulene, cadinene, caryophyllene, and caryophyllene oxide. According to some embodiments, the vaporizable formulation comprises from 50 to 90 wt % of the cannabinoid. According to some embodiments, the vaporizable formulation comprises from 55 to 85 wt %, from 60 to 80 wt %, or from 65 to 75 wt % of the cannabinoid. According to some embodiments, the vaporizable formulation comprises from 10 to 60 wt %, from 15 to 55 wt %, from 20 to 50 wt %, from 25 to 45 wt %, from 30 to 40 wt %, from 10 to 30 wt %, or from 30 to 60 wt % of the cannabinoid. According to some embodiments, the vaporizable formulation comprises from 20 to 55 wt % of the cannabinoid.

According to any one of the above embodiments, the cannabinoid is selected from the group consisting of cannabidiol (CBD), cannabidiolic acid (CBDA), tetrahydrocannabinol (THC), tetrahydrocannabinolic acid (THCA), THC acetate, cannabigerol (CBG), cannabigerolic acid (CBGA), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), tetrahydrocannabivarinic acid (THCVA), cannabidivarin (CBDV), cannabichromevarin (CBCV), cannabichromevarinic acid (CBCVA), cannabigerovarin (CBGV), cannabichromenic acid (CBCA), cannabidivarinic acid (CBDVA) cannabigerovarinic acid (CBGVA) and cannabigerol monomethyl ether (CBGM), salts thereof, derivatives thereof and mixtures of cannabinoids.

According to some embodiments, the cannabinoid is THC. According to one embodiment, the vaporizable formulation comprises from 50 to 90 wt % THC, from 3 wt % to 12 wt % of phytol and from 3 wt % to 12 wt % of bisabolol. According to another embodiment, the vaporizable formulation comprises from 3 wt % to 27 wt % of phytol and from 3 wt % to 27 wt % of bisabolol and from 60 to 90 wt % THC. According to some embodiments, the vaporizable formulation comprises from 55 to 85 wt %, from 60 to 80 wt %, or from 65 to 75 wt % THC. According to one embodiment, the formulation comprises from 55 to 80 wt % or from 60 to 80 wt % THC, from 5 to 10 wt % of natural phytol and from 5 wt % to 10 wt % of a-(−)-bisabolol.

According to other embodiments, the cannabinoid is CBD. According to some embodiments, the vaporizable formulation comprises from 30 to 90 wt % CBD, from 3 wt % to 35 wt % of phytol and from 3 wt % to 35 wt % of bisabolol. According to some embodiments, the formulation comprises from 40 to 70 wt % or from 45 to 65 wt % of CBD. According to other embodiments, the vaporizable formulation comprises from 3 wt % to 27 wt % of phytol and from 3 wt % to 27 wt % of bisabolol and from 40 to 85 wt % CBD. According to other embodiments, the formulation comprises from 45 to 85 wt %, from 50 to 80 wt %, from 55 to 75 wt % or from 60 to 70 wt % of CBD. According to other embodiments, the formulation comprises from 50 to 70 wt % of CBD. According to one embodiment, the formulation comprises from 7 to 20 wt % of bisabolol and from 7 to 20 wt % phytol. According to one embodiment, the bisabolol is α-(−)-bisabolol. According to some embodiments, the vaporizable formulation comprises from 45 to 65 wt % of CBD, from 10 to 20 wt % α-(−)-bisabolol and from 10 to 20 wt % of phytol, e.g. natural phytol. According to one embodiment, the cumulative content of bisabolol and phytol is at least 15 wt % of the formulation. According to some embodiments, the vaporizable formulation comprises from 45 to 65 wt % of CBD, from 10 to 20 wt % α-(−)-bisabolol and from 10 to 20 wt % of phytol, e.g. natural phytol, wherein the cumulative content of bisabolol and phytol is at least 15 wt % of the formulation.

According to other embodiments, the formulation comprises a combination of cannabinoids. According to one embodiment, the vaporizable formulation comprises a combination of CBD and THC. According to some embodiments, the vaporizable formulation comprises from 20 to 55 wt % of CBD, from 30 to 55 wt % of THC, from 3 wt % to 27 wt % of phytol and from 3 wt % to 27 wt % of bisabolol. According to other embodiments, the vaporizable formulation comprises from 30 to 55 wt % of CBD, from 30 to 55 wt % of THC, from 3 wt % to 27 wt % of phytol and from 3 wt % to 27 wt % of bisabolol. According to some embodiments, the molar ratio of CBD to THC is from 5:1 to 1:5. According to another embodiment, the molar ratio of CBD to THC is from 4:1 to 1:4, 3:1 to 1:3, from 2:1 to 1:2 or about 1:1. According to one embodiment, the bisabolol is α-(−)-bisabolol. According to one embodiments, the cumulative content of bisabolol and phytol is at least 10% or at least 15 wt % of the formulation.

According to another embodiment, the vaporizable formulation comprises a combination of CBD and CBDA. According to one embodiment, the vaporizable formulation comprises from 30 to 65 wt % of CBD, from 10 to 55 wt % of CBDA, from 3 wt % to 27 wt % of phytol and from 3 wt % to 27 wt % of bisabolol. According to one embodiment, the vaporizable formulation comprises from 30 to 55 wt % of CBD, from 30 to 55 wt % of CBDA, from 3 wt % to 27 wt % of phytol and from 3 wt % to 27 wt % of bisabolol. According to one embodiment, the formulation comprises from 35 to 50 wt % of CBD and from 35 to 50 wt % CBDA. According to some embodiments, the molar ratio of CBD to CBDA is from 5:1 to 1:5. According to another embodiment, the molar ratio of CBD to CBDA is from 4:1 to 1:4, 3:1 to 1:3, from 2:1 to 1:2 or about 1:1. According to one embodiment, the bisabolol is α-(−)-bisabolol. According to one embodiment, the cumulative content of bisabolol and phytol is at least 10% or at least 15 wt % of the formulation.

According to some embodiments, the cumulative concentration of the cannabinoid(s) in the vaporizable formulation is from 30 to 85 wt %. According to one embodiment, the cumulative concentration of the cannabinoid(s) in the vaporizable formulation is from 35 to 80 wt %, from 40 to 75 wt %, from 45 to 70 wt %, from 50 to 65 wt % or from 55 to 60 wt %. According to one embodiment, the cumulative concentration of the cannabinoid(s) in the vaporizable formulation is from 40 to 87 wt %. According to some embodiments, the cumulative content of bisabolol and phytol is at least 10 wt % of the formulation. According to some embodiments, the cumulative content of bisabolol and phytol is at least 15 wt % of the formulation.

According to any one of the above embodiments, weight ratio of sesquiterpene to diterpene is from 1:10 to 10:1. According to some embodiments, weight ratio of sesquiterpene to diterpene is from 1:9 to 9:1, from 1:8 to 8:1, from 1:7 to 7:1 or from 1:6 to 6:1. According to any one of the above embodiments, weight ratio of sesquiterpene to diterpene is from 1:5 to 5:1. According to another embodiment, the weight ratio between the sesquiterpene to diterpene is from 1:4 to 4:1. According to one embodiment, the weight ratio between the sesquiterpene to diterpene is from 1:3 to 3:1. According to some embodiments, the weight ratio between the sesquiterpene to diterpene is, from 1:2 to 1:2 or about 1:1. According to other embodiments, the weight ratio between the sesquiterpene to diterpene is from 1:1 to 1:5. According to one embodiment, the diterpene is phytol. According to another embodiment, the sesquiterpene is bisabolol.

According to any one of the above embodiments, the vaporizable formulation further comprises nicotine. According to some embodiments, the nicotine is provided as a tobacco extract. Thus, according to one embodiment, the vaporizable formulation further comprises a tobacco extract. According to one embodiment, the vaporizable formulation comprises from 0.1 to 7 wt %, from 0.2 to 5 wt %, from 0.3 to 3 wt %, from 1 to 3 wt % or from 0.5 to 2 wt % of nicotine or tobacco extract. The tern “nicotine” refers to the basic form and to a salt of the nicotine such as benzoic acid salt, salicylic acid, sorbic acid, benzoic acid, lauric acid, levulinic acid and malic acid. According to some embodiments, the vaporizable formulation comprises from 3 wt % to 35 wt % of a sesquiterpene and from 3 wt % to 35 wt % of a diterpene from 40 to 75 wt % of CBD and from 0.5 to 3 wt % of nicotine. According to one embodiment, the diterpene is phytol and the sesquiterpene is bisabolol. According to one embodiment, the bisabolol is α-(−)-bisabolol. According to one embodiment, the phytol is natural phytol. According to one embodiment, the bisabolol is α-(−)-bisabolol and the phytol is a natural phytol. According to some embodiments, the formulation comprises cumulatively at least 10 wt % of sesquiterpene and phytol. According to other embodiments, the formulation comprises cumulatively at least 15 wt % of sesquiterpene and phytol. According to any one of the above embodiments, the formulation further comprises an antioxidant. According to one embodiment the antioxidant is selected from citric acid, ascorbyl palmitate and a combination thereof. According to some embodiments, the antioxidant is selected from citric acid, ascorbyl palmitate, malic acid, tartaric acid oxalic acid, succinic acid and ascorbyl stearate, and any combination thereof. According to some embodiments, the formulation comprises from 0.001 to 1 wt % of the antioxidants, from 0.05 to 0.9, from 0.1 to 0.6 from 0.2 to 0.5 wt % of the antioxidant. According to some embodiments, the formulation comprises from 0.005 to 0.5 wt % of the antioxidant, from 0.01 to 0.4 wt %, from 0.03 to 0.35 wt %, from 0.05 to 0.3 wt % of the antioxidant.

According to any one of the above embodiments, the formulation further comprises an inti-irritant, such as menthol.

One of the main advantages of the vaporizable formulations of the present invention as discussed above is that the dose of the formulation obtained upon vaporing does not change upon emptying of the cartridge. Typically, when the amount of a formulation in a cartridge reduces, the vaped amount of the formulation changes and subsequently the vaped amount of the active ingredients such as cannabinoids changes as well. This effect does not occur when using the vaporizable formulation of the present invention. Thus, according to any one of the aspects and embodiments, of the present invention, the vaporizable formulation upon vaporization provides a substantially constant dose of the cannabinoid. According to some embodiments, the deviation between the administered doses of cannabinoid upon multiple vaporizations is no more than 20%. According to some embodiments, the deviation between the doses of a cannabinoid between two non-consecutive administrations via vaporization is no more than 20%. According to other embodiments, the deviation is no more than 15, 10 or 5%. In other word, the present invention provides composition which upon multiple vaporization keep administering substantially constant dose of the active ingredient.

According to any one of the above aspects and embodiments, the vaporizable composition may further comprise additional excipients. Non-limiting examples of such excipients are ethanol, ethyl acetate, propylene glycol, glycerol, tocopherol, 1,3-propane diol, MCT, coconut oil, triethyl citrate, and squalane. According to some embodiments, the additional excipients may be natural or artificial flavoring agents and antioxidants. According to some embodiments, the vaporizable composition may further comprises additional terpenes, such as menthol used, for examples as flavoring agents. According to some embodiments, the formulation may be devoid of the excipients listed above in the paragraph. According to some embodiments, the formulation is devoid of one, some or all of the excipients listed above in the paragraph. According to any one of the above embodiments, the bisabolol is α-(−)-bisabolol. According to one embodiment, the phytol is natural phytol.

According to any one of the above and below embodiments and aspects, the vaporizable formulation is devoid of crystals of cannabinoids. According to some embodiments, the vaporizable formulation comprising CBD is devoid of CBD crystals. The terms “substantially devoid”, “essentially devoid”, “devoid”, “does not include” and “does not comprise” may be used interchangeably and refer to formulation that does not include, contain or comprise a particular component, e.g. said composition comprises less than 0.1 wt %, less than 0.01 wt %, or less than 0.001 wt % of the component, specifically of cannabinoid crystals. According to any one of the above embodiments and aspects, the vaporizable formulation prevents crystallization of cannabinoids, e.g. prevent crystallization of CBD.

According to another aspect, the present invention provides a vaporizable formulation, comprising from 3 to 45 wt % of bisabolol and at least one cannabinoid. According to one embodiment, the bisabolol is a natural bisabolol, i.e. α-(−)-bisabolol.

According to one embodiment, the vaporizable formulation comprises from 5 to 30 wt % of α-(−)-bisabolol. According to another embodiment, the vaporizable formulation comprises from 5 to 30 wt % of α-(−)-bisabolol. According to a further embodiment, the vaporizable formulation comprises from 15 to 30 wt % of α-(−)-bisabolol. According to other embodiments, the formulation comprises from 15 to 25 wt %, from 20 to 30 wt % or from 15 to 25 wt % of α-(−)-bisabolol.

According to one embodiment, the bisabolol is a sole diluent.

According to some embodiments, some of the bisabolol may be replaced by one or a mixture of the sesquiterpene selected from valenciene, humulene, cadinene, caryophyllene, caryophyllene oxide. According to one embodiment, from 1 to 40% of the bisabolol is replaced by the a combination of the sesquiterpene consisting of valenciene, humulene, cadinene, caryophyllene and caryophyllene oxide. According to one embodiment, the combination comprises equal amounts of each one of the sesquiterpenes.

According to some embodiments, the vaporizable composition may further comprise additional excipients. Non-limiting examples of such excipients are ethanol, ethyl acetate, propylene glycol, glycerol, tocopherol, 1,3-propane diol, MCT, coconut oil, triethyl citrate, and squalane. According to some embodiments, the additional excipients may be natural or artificial flavoring agents and antioxidants. According to some embodiments, the vaporizable composition may further comprises additional terpenes, such as menthol used, for examples as flavoring agents. According to some embodiments, the formulation may be devoid of the excipients listed above in the paragraph. According to some embodiments, the formulation is devoid of one, some or all of the excipients listed above in the paragraph.

According to any one of the above embodiments, the cannabinoid is selected from the group consisting of cannabidiol (CBD), cannabidiolic acid (CBDA), tetrahydrocannabinol (THC), tetrahydrocannabinolic acid (THCA), THC acetate, cannabigerol (CBG), cannabigerolic acid (CBGA), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), tetrahydrocannabivarinic acid (THCVA), cannabidivarin (CBDV), cannabichromevarin (CBCV), cannabichromevarinic acid (CBCVA), cannabigerovarin (CBGV), cannabichromenic acid (CBCA), cannabidivarinic acid (CBDVA) cannabigerovarinic acid (CBGVA) and cannabigerol monomethyl ether (CBGM), salts thereof, derivatives thereof and mixtures of cannabinoids.

According to one embodiment, the cannabinoid is selected from THC, CBD, a combination of THC and CBD or a combination of CBD and CBDA. According to some embodiments, the vaporizable formulation comprises from 50 to 90 wt % of a cannabinoid. According to some embodiments, the vaporizable formulation comprises from 55 to 85 wt %, from 60 to 80 wt %, or from 65 to 75 wt % of cannabinoids, such as THC, CBD, combination of THC and CBD or a combination of CBD and CBDA.

According to some embodiments, the vaporizable formulation comprises from 5 to 45 wt % bisabolol and from 55 to 87 wt % of CBD. According to some embodiments, the vaporizable formulation comprises from 5 to 25 wt % bisabolol and from 55 to 87 wt % of CBD. According to some embodiments, the vaporizable formulation comprises from 5 to 25 wt % bisabolol and from 60 to 87 wt % of CBD. According to some embodiments, the vaporizable formulation comprises from 5 to 25 wt % bisabolol and from 65 to 85 wt % of CBD. According to some embodiments, the vaporizable formulation comprises from 5 to 25 wt % bisabolol and from 70 to 80 wt % of CBD. According to some embodiments, the vaporizable formulation comprises from 10 to 20 wt % bisabolol and from 70 to 85 wt % or from 60 to 85 wt % of CBD. According to some embodiments, the vaporizable formulation comprises from 5 to 15 or from 20 to 25 wt % bisabolol and from 65 to 85 wt % or from 70 to 85 wt % of CBD. According to other embodiments, the vaporizable formulation comprises from 10 to 45 wt % of bisabolol and from 65 to 87 wt % of CBD. According to some embodiments, the vaporizable formulation comprises from 15 to 40 wt % of bisabolol and from 65 to 85 wt % of CBD. According to some embodiments, the vaporizable formulation comprises from 15 to 40 wt % of bisabolol and from 40 to 70 wt % of CBD. According to some embodiments, the vaporizable formulation comprises from 15 to 40 wt % of bisabolol, and from 70 to 80 wt %, from 75 to 85 wt %, from 65 to 85 wt %, from 70 to 85 wt %, from 70 to 80, or from 75 to 85 wt % of CBD. According to some embodiments, the vaporizable formulation comprises from 20 to 40 wt % of bisabolol, and from 70 to 80 wt %, from 75 to 85 wt %, from 65 to 85 wt %, from 70 to 85 wt %, from 70 to 80 wt %, or from 75 to 87 wt % of CBD. According to some embodiments, the vaporizable formulation comprises from 25 to 45 wt % of bisabolol, and from 70 to 80 wt %, from 75 to 85 wt %, from 65 to 85 wt %, from 70 to 85 wt %, from 70 to 80, or from 75 to 87 wt % of CBD. Such composition provides stable composition of CBD using only one terpene. According to some embodiments, the vaporizable formulation comprises from 30 to 40 wt % of bisabolol, and from 70 to 80 wt %, from 75 to 85 wt %, from 65 to 85 wt %, from 70 to 85 wt %, from 70 to 80, or from 75 to 87 wt % of CBD. According to some embodiments, the vaporizable formulation comprises from 15 to 35 wt % of bisabolol, and from 70 to 80 wt %, from 75 to 85 wt %, from 65 to 85 wt %, from 70 to 85 wt %, from 70 to 80, or from 75 to 87 wt % of CBD. According to some embodiments, the vaporizable formulation comprises from 5 to 45 wt %, from 10 to 40, from 15 to 35, from 20 to 35, from 5 to 30 wt % or from 5 to 15 wt % of bisabolol. According to some embodiments, the bisabolol is α-(−)-bisabolol.

According to any one of the above embodiments, the vaporizable formulation further comprises nicotine. According to some embodiments, the nicotine is synthetic. According to other embodiments, the nicotine is provided as a tobacco extract or purified from tobacco. Thus, according to one embodiment, the vaporizable formulation further comprises a tobacco extract. According to one embodiment, the vaporizable formulation comprises from 0.1 to 7 wt %, from 0.2 to 5 wt %, from 0.3 to 3, from 2 to 4, from 1 to 3 or from 0.5 to 2 wt % of nicotine or tobacco extract. The term “nicotine” refers to the basic form and to a salt of the nicotine such as benzoic acid salt, salicylic acid, sorbic acid, benzoic acid, lauric acid, levulinic acid and malic acid. According to some embodiments, the vaporizable formulation comprises from 5 to 45 wt % of bisabolol, from 40 to 85 wt % of CBD and from 0.5 to 3 wt % of nicotine. According to some embodiments, the vaporizable formulation comprises from 10 to 45 wt % of bisabolol, from 40 to 75 wt % of CBD and from 0.5 to 3 wt % of nicotine. According to some embodiments, the vaporizable formulation comprises from 5 to 45 wt % of bisabolol, from 40 to 75 wt % of CBD and from 0.5 to 3 wt % of nicotine. According to one embodiment, the bisabolol is α-(−)-bisabolol. According to other embodiments, the vaporizable formulation comprises from 5 to 45 wt % of bisabolol, from 50 to 85 wt % of CBD and from 0.5 to 3 wt % of nicotine. According to other embodiments, the vaporizable formulation comprises from 5 to 45 wt % of bisabolol, from 60 to 85 wt % of CBD and from 0.5 to 3 wt % of nicotine. According to some embodiments, the vaporizable formulation comprises from 5 to 45 wt % of bisabolol, from 40 to 85 wt % of CBD and from 0.5 to 3 wt % of nicotine. According to some embodiments, the vaporizable formulation comprises from 5 to 25 wt % of bisabolol, from 60 to 85 wt % of CBD and from 0.5 to 3 wt % of nicotine. According to some embodiments, the vaporizable formulation comprises from 15 to 45 wt % of bisabolol, from 50 to 85 wt % of CBD and from 0.5 to 3 wt % of nicotine. According to other embodiments, the vaporizable formulation comprises from 10 to 25 wt % of bisabolol, from 70 to 85 wt % of CBD and from 0.5 to 3 wt % of nicotine. According to some embodiments, the vaporizable formulation comprises from 15 to 45 wt % of bisabolol, from 70 to 85 wt % of CBD and from 0.5 to 3 wt % of nicotine. According to some embodiments, the vaporizable formulation comprises from 30 to 40 wt % of bisabolol, from 70 to 80 wt %, from 75 to 85 wt %, from 65 to 85 wt %, from 70 to 85 wt %, from 70 to 80, or from 75 to 87 wt % of CBD and from 0.5 to 3 wt % of nicotine. According to some embodiments, the vaporizable formulation comprises from 15 to 35 wt % of bisabolol, from 70 to 80 wt %, from 75 to 85 wt %, from 65 to 85 wt %, from 70 to 85 wt %, from 70 to 80, or from 75 to 87 wt % of CBD and from 0.5 to 3 wt % of nicotine. According to some embodiments, the vaporizable formulation comprises from 5 to 45 wt %, from 10 to 40 wt %, from 15 to 35 wt %, from 20 to 35 wt %, from 5 to 30 wt % or from 5 to 15 wt % of bisabolol and from 0.5 to 3 wt % of nicotine. According to some embodiments, the bisobolol is α-(−)-bisabolol.

According to one embodiment, the bisabolol is α-(−)-bisabolol. According to some embodiments, the vaporizable formulation is devoid of CBD crystals. According to some embodiments, the formulation prevents crystallization of CBD.

According to any one of the above embodiments, the formulation further comprises an antioxidant. According to one embodiment the antioxidant is selected from citric acid, ascorbyl palmitate and a combination thereof According to some embodiments, the antioxidant is selected from citric acid, ascorbyl palmitate, malic acid, tartaric acid oxalic acid, succinic acid and ascorbyl stearate, and any combination thereof. According to some embodiments, the formulation comprises from 0.001 to 1 wt % of the antioxidants, from 0.05 to 0.9, from 0.1 to 0.6 from 0.2 to 0.5 wt % of the antioxidant. According to some embodiments, the formulation comprises from 0.005 to 0.5 wt % of the antioxidant, from 0.01 to 0.4 wt %, from 0.03 to 0.35 wt %, from 0.05 to 0.3 wt % of the antioxidant.

According to any one of the above embodiments, the formulation further comprises an inti-irritant, such as menthol.

According to any one of the above aspects and embodiments, the vaporizable formulation is prepared by admixing at least one cannabinoid with a stock composition of the present invention comprising a sesquiterpene and a diterpene, wherein the cumulative concentration of the sesquiterpene and diterpene in the stock composition is from 70 wt % to 100 wt %, and wherein the weight ratio of the sesquiterpene to diterpene is from 1:10 to 10:1. According to some embodiments, the vaporizable formulation is prepared by admixing at least one cannabinoid with a stock composition of the present invention comprising a sesquiterpene and a phytol, wherein the cumulative concentration of the sesquiterpene and phytol in the stock composition is from 70 wt % to 100 wt %, and wherein the weight ratio of the sesquiterpene to phytol is from 1:10 to 10:1. According to some embodiments, the cumulative content of a sesquiterpene and diterpene is at least 10 wt % of the vaporizable formulation. According to some embodiments, the cumulative content of a bisabolol and a phytol is at least 10 wt % of the vaporizable formulation. According to another embodiment, the vaporizable formulation is prepared by admixing at least one cannabinoid with concentrated sesquiterpene and with concentrated diterpene to obtain the desired defined concentrations. According to some embodiments, the vaporizable formulation is prepared by admixing cannabis oil comprising at least one cannabinoid with a composition of the present invention comprising a sesquiterpene and a diterpene. According to some embodiments, the cannabis oil further comprises one or more sesquiterpene and/or diterpene. According to some embodiments, the cumulative content of a sesquiterpene such as a bisabolol and of diterpene such as a phytol is at least 10 wt % of the vaporizable formulation. According to other embodiments, the cumulative content of a sesquiterpene such as a bisabolol and of diterpene such as a phytol is at least 15 wt % of the resulted vaporizable formulation. According to one embodiment the cumulative content of a sesquiterpene such as a bisabolol and of diterpene such as a phytol is at least 20 wt % of the resulted vaporizable formulation.

The formulation of the present invention may be prepared by mixing the desired cannabinoids fraction(s) with the desired terpenes in closed container protected from light and then used for filling the vaporizer cartridges. The composition of the present invention may be prepared by mixing the purified terpenes in the desired proportions.

According to another aspect, the present invention provides a container comprising the stock composition of the present invention. Any terms and embodiments related to the stock composition of the present invention apply herein as well.

According to another aspect, the present invention provides a container comprising the vaporizable formulation of the present invention. According to some embodiments, the container is suitable for use with a vaporizer, for example with using cartomizers, atomizers or clearomizer type vaporizer. According to one embodiment, the container is a cartridge. According to some embodiments, the container, cartridge or tank containing the vaporizable formulation is a disposable or refillable container. According to other embodiments, the container is suitable or configured to use with a vaporizer utilizing a wick and coil heating element or utilizing a heating chamber such as ceramic heating chamber or titanium/quartz heating chamber. According to some embodiments, the container is a wick and coil container or a ceramic container. According to some embodiments, the ceramic container is a VapePod container using the technology called CCELL Ceramic Cell Technology. In an ordinary atomizer coil resides on the surface of the wick causing inconsistent heating. Additionally, ordinary wick designs cause a reduced flow to the heating coil. The heating coil which is embedded within the ceramic core ensures the atomizer is uniformly heated. According to some embodiments, the container is a wick and coil container or a ceramic container.

The term “vaporizer” as used herein refers to a device used to vaporize a composition, in particular the composition of the present invention. Example of such vaporizer are cartomizers, atomizers and clearomizer. According to some embodiments, the vaporizer utilizes a wick and coil heating element or a heating chamber such as ceramic heating chamber or titanium/quartz heating chamber. According to some embodiments, the container, cartridge or tank containing the formulation is a disposable or refillable container, cartridge or tank. According to some embodiments, the vaporizer is an extraction vaporizer.

According to some embodiments, the present invention provides a container comprising the vaporizable formulation of the present invention comprising from 5 to 70 wt % or from 5 to 60 wt % of the stock composition according to any one of the above embodiments and aspects and at least one cannabinoid. Thus, in some embodiments, the present invention provides a container comprising a vaporizable formulation comprising from 5 to 60 wt % of the stock composition of the present invention comprising a sesquiterpene and a diterpene, and at least one cannabinoid, wherein the cumulative concentration of the sesquiterpene and diterpene in the stock composition is from 70 wt % to 100 wt %, and wherein the weight ratio of the sesquiterpene to diterpene in the stock composition is from 1:10 to 10:1. According to some embodiments, the cannabinoid is CBD. According to some embodiments, the vaporizable formulation comprises from 30 to 90 wt % CBD, from 3 wt % to 35 wt % of phytol and from 3 wt % to 35 wt % of bisabolol. According to some embodiments, the formulation comprises from 40 to 70 wt % or from 45 to 65 wt % of CBD. According to other embodiments, the formulation comprises from 45 to 85 wt %, from 50 to 80 wt %, from 55 to 75 wt % or from 60 to 70 wt % of CBD. According to one embodiment, the formulation comprises from 7 to 20 wt % of bisabolol and from 7 to 20 wt % phytol. According to one embodiment, the bisabolol is α-(−)-bisabolol. According to some embodiments, the vaporizable formulation comprises from 45 to 65 wt % of CBD, from 10 to 20 wt % α-(−)-bisabolol and from 10 to 20 wt % of phytol, e.g. natural phytol. According to one embodiments, the cumulative content of bisabolol and phytol is at least 15 wt % of the formulation. According to some embodiments, the vaporizable formulation comprises from 45 to 65 wt % of CBD, from 10 to 20 wt % α-(−)-bisabolol and from 10 to 20 wt % of phytol, e.g. natural phytol, wherein the cumulative content of bisabolol and phytol is at least 15 wt % of the formulation. According to another embodiment, the vaporizable formulation comprises a combination of CBD and CBDA. According to one embodiment, the vaporizable formulation comprises from 30 to 65 wt % of CBD, from 10 to 55 wt % of CBDA, from 3 wt % to 27 wt % of phytol and from 3 wt % to 27 wt % of bisabolol. According to one embodiment, the vaporizable formulation comprises from 30 to 55 wt % of CBD, from 30 to 55 wt % of CBDA, from 3 wt % to 27 wt % of phytol and from 3 wt % to 27 wt % of bisabolol. According to one embodiment, the formulation comprises from 35 to 50 wt % of CBD and from 35 to 50 wt % CBDA. According to some embodiments, the molar ratio of CBD to CBDA is from 5:1 to 1:5. According to another embodiment, the molar ratio of CBD to CBDA is from 4:1 to 1:4, 3:1 to 1:3, from 2:1 to 1:2 or about 1:1. According to one embodiment, the bisabolol is α-(−)-bisabolol. According to some embodiments, the cumulative content of terpenes, such as sesquiterpene and diterpenes is at least 10 wt % or at least 15 wt % or at least 20 wt % of the vaporizable formulation.

According to some embodiments, the composition further comprises nicotine, e.g. about 0.5 to 4 wt % of nicotine or tobacco extract. According to some embodiments, the container comprises a vaporizable formulation comprises from 3 wt % to 35 wt % of a sesquiterpene such as a bisabolol and from 3 wt % to 35 wt % of a diterpene such as a phytol, from 40 to 75 wt % of CBD and from 0.5 to 3 wt % of nicotine.

According to another aspect, the vaporizable formulation of the present invention is for use as a medicament. According to some embodiments, the vaporizable formulation of the present invention is for use in treating a disease or a disorder treatable with the cannabinoid. The vaporizable formulation is as defined in any one of the above embodiments and aspects, Thus, according to some embodiments, vaporizable formulation comprising at least one cannabinoid and from 5 to 60 wt % of the stock composition of the present invention and at least one cannabinoid, is for use in treating a disease or a disorder that may be treated with the at least one cannabinoid, wherein the stock composition comprises a sesquiterpene and a diterpene, wherein the cumulative concentration of the sesquiterpene and diterpene in the stock composition is from 70 wt % to 100 wt %, and wherein the weight ratio of the sesquiterpene to diterpene is from 1:10 to 10:1, According to some embodiments, the vaporizable formulation comprises from 50 to 90 wt % of the cannabinoid. According to some embodiments, the vaporizable formulation comprises from 55 to 85 wt %, from 60 to 80 wt %, or from 65 to 75 wt % of the cannabinoid. According to some embodiments, the cannabinoid is CBD. According to some embodiments, the vaporizable formulation comprises from 10 to 70 wt % of the terpenes.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

According to some embodiments, the disease or disorder may be, for example, neurological conditions and psychiatric disorders. According to some embodiments, the neurological conditions and psychiatric disorders are selected from anxiety disorders, PTSD, addiction, alcoholism, OCD, amyotrophic lateral sclerosis (ALS), arachnoid cysts, attention deficit/hyperactivity disorder (ADHD), autism, bell's palsy, bipolar disorder, brain/spinal cord tumors, carpal tunnel syndrome, catalepsy, cervical spondylosis, depression, dizziness, encephalitis, epidural abscess, epilepsy/seizures, extradural hemorrhage, sleep disorders, Guillain-Barre syndrome, headache, hematoma, infection, locked-in-syndrome, meningitis, migraine, multiple sclerosis, myelopathy, neuralgia, neurodegenerative disorders such as Alzheimer's disease, Huntington's disease and Parkinson's disease; peripheral neuropathy, polio, spinal cord injury, stroke, subarachnoid hemorrhage, subdural hemorrhage, Tourette's, transient ischemic attack (TIA), and schizophrenia/mental disorders. According to some embodiments, the disease or disorder is selected from anxiety, PTSD, pain, epilepsy, depression, migraines, cluster headache, bipolar disorder, multiple sclerosis, Parkinson's disease, Alzheimer's disease, Huntington disease, and Tourette syndrome. According to other embodiments, the disease or disorder is selected from anorexia, arthritis, asthma, atherosclerosis, atrophie blanche, back pain, cancer such as breast, colorectal, glioma/brain, leukemia, skin, lung, melanoma, oral, pancreatic, prostate, skin and testicular cancer, cerebral palsy, chronic cystitis, chronic pain, chronic stress, colon cancer, COPD, Crohn's disease, dermatitis, diabetes, disc degeneration, dystonia, endocannabinoid deficiency, fibromyalgia, glaucoma, heart disease, hepatitis, herpes, hiccups, HIV/AIDS, idiopathic intracranial hypertension, liver disease, lupus, malaria, Meige's syndrome, migraines, muscular dystrophy, nausea & vomiting, neuropathic pain, obesity, osteoporosis, painful bladder syndrome, pancreatic cancer, pruritis, sickle cell disease, spasticity, spinal cord injury, stroke, substance abuse, and Wilson's disease.

According to some embodiments, the vaporizable formulation of the present invention comprising nicotine is for use in treatment of nicotine addiction. According to some embodiments, the vaporizable formulation of the present invention comprising CBD and nicotine is for use in treatment of smoking addiction. The term comprises also treatment of tobacco smoking or e-cigarette smoking addiction. According to some embodiments, the vaporizable formulation of the present invention comprising nicotine is for use in treatment of tobacco smoking addiction. According to one embodiment, the formulation comprising from 10 to 60 wt % of the stock composition comprising a sesquiterpene and a diterpene, from 40 to 75 wt % of CBD and from 0.5 to 5 wt % of nicotine, is for use in treating nicotine addiction and/or tobacco smoking addiction, wherein the cumulative concentration of the sesquiterpene and diterpene in the stock composition is from 70 wt % to 100 wt % and the weight ratio of the sesquiterpene to diterpene is from 1:10 to 10:1. According to another embodiment, the vaporizable formulation comprising from 3 wt % to 35 wt % of a sesquiterpene and from 3 wt % to 35 wt % of a diterpene from 40 to 75 wt % of CBD and from 0.5 to 5 wt % of nicotine is for use in treating nicotine addiction and/or tobacco smoking addiction. According to some embodiments, the vaporizable formulation comprising from 3 to 45 wt % of bisabolol, from 40 to 87 wt % of CBD and from 0.5 to 5 wt % of nicotine is for use in treatment of nicotine addiction and/or tobacco smoking addiction. According to some embodiments, the bisabolol is α-(−)-bisabolol. According to one embodiment, the phytol is natural phytol. According to some embodiments, the cumulative content of sesquiterpene and diterpenes such as bisabolol and phytol is at least 10 wt % of the vaporizable formulation. According to some embodiments, the cumulative content of sesquiterpene and diterpenes such as bisabolol and phytol is at least 15, at least 20, or at least 25 wt % of the vaporizable formulation. According to another embodiment, the cumulative content of bisabolol and phytol is at least 10 wt % of the formulation.

The terms “treatment of addiction to nicotine” and “treatment of nicotine addiction” are used herein interchangeably and include both partial and complete alleviation of addiction to nicotine, nicotine comprising products or tobacco consumption e.g. tobacco smoking, and may be replaced by any one of these terms. Thus, in respect of tobacco products, as well as the cessation of the activity, for example smoking, this also includes reducing the level or frequency of such activity e.g. reduction of the number of cigarettes smoked in a given period. In respect of other nicotine-containing products, treatment will also involve both cessation of, and a reduction in the level of, usage of such products. The term includes discontinuation of use, decrease of use, prevention of relapse, reduction in severity of use, reduction in desire and/or cravings, reduction in withdrawal symptoms, reduction in desire to use addictive products, increase is aversion to the addictive products, and loss of interest in the addictive product. As used herein, the term “addiction” shall include abuse, dependency, craving, including, but not limited to post-deprivation craving, post-withdrawal craving, relapse craving, withdrawal, and related disorders.

According to some embodiments, the vaporizable composition is administered by vaporization by using a suitable device for vaporization. According to one embodiment, the administered amount of the composition is from 0.8 to 2 mg/puff.

The method for treatment of nicotine and/or tobacco smoking addiction comprises administering the vaporizable formulation comprising nicotine from 1 to 5 inhalation(s) each time there is a need for a cigarette. According to some embodiments, the method is for treatment of smoking addictions.

According to another aspect, the present invention provides a method of treating a disease, disorder, or condition treatable with a cannabinoid, the method comprises administration via vaporization the vaporizable formulation of the present invention. According to one embodiment, the condition is nicotine addiction. According to one embodiment, the condition is smoking addiction.

According to another aspect, the present invention provides use of the stock composition of the present invention and a cannabinoid for preparation of a medicament of treatment a disease treatable with the cannabinoid. According to another aspect, the present invention provides use of the stock composition of the present invention, a cannabinoid such as CBD and nicotine for preparation of a medicament for treatment of nicotine addiction and/or tobacco smoking addiction.

According to any embodiments and aspects of the present invention, the cannabinoids used in the formulations of the present invention may be obtained by any known method. According to some embodiments, a cannabis extract may be used. The extract of cannabis of the present invention may be obtained by any known method of extraction. According to some embodiments, a cannabinoid oil is used. Alternatively, distilled cannabinoid extract or oil or cannabinoid crystals may be used.

According to another aspect, the present invention provides a kit comprising (i) the stock composition of the present invention (ii) a composition comprising at least one cannabinoid; and (iii) instructions to use said compositions for preparation of a vaporizable formulation.

According to one embodiment, the composition comprising at least one cannabinoid is a composition, oil, extract or a distillate thereof comprising a cannabinoid.

According to one embodiments, the stock composition comprises a sesquiterpene and a diterpene, wherein the cumulative concentration of the sesquiterpene and diterpene is from 70 wt % to 100 wt %, and wherein the weight ratio of the sesquiterpene to diterpene is from 1:10 to 10:1. According to some embodiments, the cumulative content of the sesquiterpene and phytol in the composition is from 75 to 95 wt % or from 80 to 90 wt %. According to one embodiment, the cumulative content of the sesquiterpene and phytol in the composition is from 85 to 100 wt % or from 90 to 100 wt %.

According to another embodiment, the cumulative content of the sesquiterpene and phytol in the composition is from 92 to 100 wt %, from 95 to 100 wt %, or from 97 to 100 wt %. According to another embodiment, the cumulative content of the sesquiterpene and phytol in the composition is from 80 to 95 wt % from 85 to 95 wt % or from 85 to 98 wt %. According to one embodiment, the sesquiterpene is bisabolol. According to some embodiments, the molar ratio of sesquiterpene and diterpene, is from 1:3 to 3:1, from 1:2 to 2:1, or about 1:1. According to some embodiments, the molar ratio of bisabolol, e.g. α-(−)-bisabolol and natural phytol is selected from 1:2, 2:1 and 1:1. According to some embodiments, the cumulative content of the natural bisabolol and phytol in the composition is about 90, 91, 92, 93, 94, 95, 96, 97, 98, 99 or about 99 wt %.

According to some embodiments, the cannabinoid is selected from CBD, THC, a combination of CBD and THC or a combination of CBD and CBDA.

According to another aspect, the present invention provides a vaporizable formulation comprising from 0.5 to 10 wt % of nicotine, a cannabinoid and at least one diluent. Such compositions allows administering a consistent, reproducible and controllable amount of a cannabinoid such as CBD and nicotine via vaporization and specifically after a plurality of administrations. Typically, after several administration the amount/concentration of administered CBD and nicotine start to vary in comparison to the initial dose. The composition of the present invention provides constant dose of these compounds with minimal variability.

According to some embodiments, the diluent has a boiling point of at least 140° C. According to some embodiments, the diluent has a boiling point of at least 150° C. According to some embodiments, the diluent has a boiling point of at least 200° C. According to some embodiments, the diluent has a boiling point of at least 230° C., at least 250° C., at least 275° C. or at least 300° C. According to some embodiments, the diluent has point of at least 290° C. According to some embodiments, the diluent has a boiling point of 140° C. or more. According to some embodiments, the diluent has a boiling point of 150° C. or more. According to some embodiments, the diluent has a boiling point of 230° C. or more. According to some embodiments, the diluent has a boiling point of 250° C. or more. According to some embodiments, the diluent has a boiling point of 275° C. or more. According to some embodiments, the diluent has a boiling point of 300° C. or more. According to some embodiments, the diluent has point of at least 350° C. As used herein, boiling point referred herein is measured at atmospheric pressure. According to some embodiments, all diluent of the vaporizable formulation ha a boiling point of at least 200° C. or as describe above.

According to some embodiments, the vaporizable formulation comprises from 5 to 50 wt % of the diluent or of the mixture of diluents. According to some embodiments, the formulation comprises from 10 to 45 wt %, from 15 to 40 wt % or from 20 to 30 wt % of the diluent or of a mixture of diluents. According to some embodiments, the vaporizable formulation comprises from 20 to 50 wt % of the diluent or of the mixture of diluents. According to some embodiments, the vaporizable formulation comprises from 25 to 45 wt % of the diluent or of the mixture of diluents. According to some embodiments, the vaporizable formulation comprises from 25 to 40 wt % of the diluent or of the mixture of diluents. According to some embodiments, the vaporizable formulation comprises from 25 to 35 wt % of the diluent or of the mixture of diluents. According to some embodiments, the formulation comprises from 5 to 25 wt % of the diluent or of the mixture of diluents. According to some embodiments, the formulation comprises from 5 to 20 wt % or from 5 to 15 wt % of the diluent or of the mixture of diluents. According to some embodiments, the diluent is a bisabolol. According to some embodiments, the diluent is alpha-bisabolol.

According to other embodiments, the formulation comprises from 50 to 95 wt % of the diluent or of a mixture of diluents. According to some embodiments, the formulation comprises from 55 to 90 wt %, from 60 to 85 wt % or from 65 to 80 wt % of the diluent or a mixture of diluents. According to one embodiment, the formulation comprises from 10 to 95 wt % or from 70 to 95 wt % of the diluent or a mixture of diluents. According to one embodiment, the formulation comprises from 60 to 90 wt % of the diluent or a mixture of diluents. According to one embodiment, the formulation comprises from 60 to 85 wt % of the diluent or a mixture of diluents. According to one embodiment, the formulation comprises from 65 to 85 wt % of the diluent or a mixture of diluents. According to one embodiment, the formulation comprises from 75 to 85 wt % of the diluent or a mixture of diluents. According to one embodiment, the formulation comprises from 70 to 90 wt % of the diluent or a mixture of diluents. According to some embodiments, the diluent is medium chain triglycerides (MCT). According to some embodiments, the diluent is selected from natural bisabolol, phytol, sesquiterpenes, diterpenes, medium chain triglycerides (MCT), glyceryl trioctanoate, glyceryl tridecanoate, squalane, triethyl citrate, PEG and any combination thereof. According to some embodiments, the diluent is (C8-C12) MCT. According to one embodiment, the diluent is glyceryl trioctanoate. According to some embodiments, the diluent is a bisabolol. According to some embodiments, the diluent is a α-(−)-bisabolol. According to one embodiment, the bisabolol is a sole diluent. According to some embodiments, the content of bisabolol is defined in any one of the above aspects and embodiments.

According some embodiments, the present invention provides a vaporizable formulation, comprising from 0.5 to 10 wt % of nicotine, from 3 to 45 wt % of bisabolol and at least one cannabinoid. According to one embodiment, the bisabolol is a natural bisabolol, i.e. α-(−)-bisabolol. According to one embodiment, the vaporizable formulation comprises from 5 to 30 wt % of α-(−)-bisabolol. According to one embodiment, the vaporizable formulation comprises from 5 to 40 wt % of α-(−)-bisabolol. According to one embodiment, the vaporizable formulation comprises from 15 to 35 wt % of α-(−)-bisabolol. According to another embodiment, the vaporizable formulation comprises from 5 to 30 wt % of α-(−)-bisabolol. According to a further embodiment, the vaporizable formulation comprises from 15 to 30 wt % of α-(−)-bisabolol. According to a further embodiment, the vaporizable formulation comprises from 20 to 40 wt % of α-(−)-bisabolol. According to a further embodiment, the vaporizable formulation comprises from 25 to 35 wt % of α-(−)-bisabolol. According to other embodiments, the formulation comprises from 15 to 25 wt %, from 20 to 30 wt % or from 15 to 25 wt % of α-(−)-bisabolol. According to other embodiments, the formulation comprises from 5 to 20 wt % or from 5 to 15 wt % of α-(−)-bisabolol. According to one embodiment, the vaporizable formulation comprises from 20 to 40 wt % of α-(−)-bisabolol. According to some embodiments, some of the bisabolol may be replaced by one or a mixture of the sesquiterpene selected from valenciene, humulene, cadinene, caryophyllene, caryophyllene oxide. According to one embodiment, from 1 to 40% of the bisabolol is replaced by a combination of the sesquiterpene consisting of valenciene, humulene, cadinene, caryophyllene and caryophyllene oxide. According to one embodiment, the combination comprises equal amounts of each one of the sesquiterpenes.

According to some embodiments, the vaporizable formulation comprises from 3 wt % to 35 wt % of a sesquiterpene and from 3 wt % to 35 wt % of a diterpene, from 40 to 75 wt % of CBD and from 0.5 to 3 wt % of nicotine. According to one embodiment, the diterpene is phytol and the sesquiterpene is bisabolol. According to one embodiment, the bisabolol is α-(−)-bisabolol. According to one embodiment, the phytol is natural phytol. According to one embodiment, the bisabolol is α-(−)-bisabolol and the phytol is a natural phytol.

According to some embodiments, the formulation comprises from 70 to 90 wt % of the diluent. According to another embodiment, the formulation comprises from 70 to 90 wt % of the C8-C12 MCT. According to yet another embodiment, the formulation comprises from 70 to 90 wt % of glyceryl tridecanoate. According to one embodiment, the formulation comprises from 60 to 90 wt % of glyceryl tridecanoate. According to one embodiment, the formulation comprises from 60 to 85 wt % of glyceryl tridecanoate. According to one embodiment, the formulation comprises from 65 to 85 wt % of glyceryl tridecanoate. According to one embodiment, the formulation comprises from 75 to 85 wt % of glyceryl tridecanoate. According to one embodiment, the formulation comprises from 70 to 90 wt % of glyceryl tridecanoate.

According to one embodiment, the formulation comprises from 60 to 90 wt % of C8-C12 MCT. According to one embodiment, the formulation comprises from 60 to 85 wt % of C8-C12 MCT. According to one embodiment, the formulation comprises from 65 to 85 wt % of C8-C12 MCT. According to one embodiment, the formulation comprises from 75 to 85 wt % of C8-C12 MCT. According to one embodiment, the formulation comprises from 70 to 90 wt % of C8-C12 MCT.

According to some embodiments, the present invention provides a vaporizable formulation comprising from 0.5 to 10 wt % of nicotine, a cannabinoid and a combination of diluent. According to some embodiments, the at least one diluent has a boiling point of at least 140° C. or at least 200° C. According to some embodiments, the combination of diluents is a combination as defined in any one of the above aspects and embodiments. According to some embodiments, the vaporizable formulation comprising from 3 wt % to 35 wt % of a sesquiterpene and from 3 wt % to 35 wt % of a diterpene. According to one embodiment, the diterpene is phytol. According to another embodiment, the sesquiterpene is bisabolol. According to one embodiment, the diterpene is phytol and the sesquiterpene is bisabolol. According to some embodiments, vaporizable formulation comprising from 3 wt % to 35 wt % of a bisabolol and from 3 wt % to 35 wt % of a phytol. According to one embodiment, the bisabolol is α-(−)-bisabolol. According to one embodiment the cumulative content of sesquiterpene and phytol is at least 15 wt % of the formulation. According to one embodiment, the vaporizable formulation comprises from 5 to 30 wt %, from 5 to 20 wt %, from 5 to 15 wt %, from 10 to 25 wt %, from 15 to 20 wt % or from 10 to 20 wt % of sesquiterpene such as bisabolol. According to another embodiment, the vaporizable formulation comprises from 5 to 20 wt %, from 5 to 15 wt %, from 5 to 30 wt %, from 10 to 25 wt %, from 15 to 20 wt % or from 10 to 20 wt % of diterpene such as phytol. According to yet another embodiment, the vaporizable formulation comprises from 5 to 30 wt %, from 10 to 25 wt %, from 5 to 15 wt %, from 10 to 25 wt %, from 15 to 20 wt % of the sesquiterpene such as bisabolol and from 5 to 30 wt %, from 5 to 15 wt %, from 10 to 25 wt %, from 10 to 25 wt %, from 15 to 20 wt % of the diterpene such as phytol. According to any one of the above embodiments, weight ratio of sesquiterpene to diterpene is from 1:10 to 10:1. According to some embodiments, weight ratio of sesquiterpene to diterpene is from 1:9 to 9:1, from 1:8 to 8:1, from 1:7 to 7:1 or from 1:6 to 6:1. According to any one of the above embodiments, weight ratio of sesquiterpene to diterpene is from 1:5 to 5:1. According to another embodiment, the weight ratio between the sesquiterpene to diterpene is from 1:4 to 4:1. According to one embodiment, the weight ratio between the sesquiterpene to diterpene is from 1:3 to 3:1. According to some embodiments, the weight ratio between the sesquiterpene to diterpene is, from 1:2 to 1:2 or about 1:1. According to other embodiments, the weight ratio between the sesquiterpene to diterpene is from 1:1 to 1:5. According to one embodiment, the diterpene is phytol. According to another embodiment, the sesquiterpene is bisabolol. According to some embodiments, the cumulative content of sesquiterpene and diterpene is from 10 to 60 wt % of the vaporizable formulation. According to some embodiments, the cumulative content of sesquiterpene and diterpene is from 15 to 55 wt % of the vaporizable formulation. According to some embodiments, the cumulative content of sesquiterpene and diterpene is from 20 to 50 wt % of the vaporizable formulation. According to some embodiments, the cumulative content of sesquiterpene and diterpene is from 10 to 40 wt % of the vaporizable formulation. According to some embodiments, the cumulative content of sesquiterpene and diterpene is from 25 to 45 wt %, from 30 to 40 wt % of the vaporizable formulation. According to some embodiments, the cumulative content of sesquiterpene and diterpene is from 15 to 55 wt % of the vaporizable formulation. According to some embodiments, the cumulative content of sesquiterpene and diterpene is from 10 to 30 wt %, or from 20 to 60 wt %, or from 15 to 45 wt % or from 30 to 60 wt % of the vaporizable formulation. According to some embodiments, the cumulative content of sesquiterpene and diterpene is from 10 to 40 wt %. According to other embodiments, the cumulative content of sesquiterpene and diterpene is from 20 to 50 wt %. According to some embodiments, the cumulative content of sesquiterpene and phytol is from 10 to 40 wt %. According to some embodiments, the cumulative content of sesquiterpene and phytol is from 20 to 50 wt %. According to some embodiments, the cumulative content of sesquiterpene and phytol is from 10 to 60 wt %, from 15 to 55 wt %, from 20 to 50 wt %, from 10 to 40 wt %, from 25 to 45 wt %, from 30 to 40 wt %, from 10 to 30 wt %, or from 20 to 60 wt %, or from 15 to 45 wt % or from 30 to 60 wt % of the vaporizable formulation. According to some embodiments, the cumulative content of sesquiterpene and phytol is from 15 to 40 wt % or from 15 to 35 wt % of the vaporizable formulation. According to some embodiments, the cumulative content of bisabolol and phytol is from 10 to 60 wt %, from 15 to 55 wt %, from 20 to 50 wt %, from 10 to 40 wt %, from 25 to 45 wt %, from 30 to 40 wt %, from 10 to 30 wt %, or from 20 to 60 wt %, or from 15 to 45 wt % or from 30 to 60 wt % of the vaporizable formulation. According to some embodiments, the cumulative content of bisabolol and phytol is from 15 to 40 wt % or from 15 to 35 wt % of the vaporizable formulation.

According to any some embodiments, the cannabinoid is selected from the group consisting of cannabidiol (CBD), cannabidiolic acid (CBDA), tetrahydrocannabinol (THC), tetrahydrocannabinolic acid (THCA), THC acetate, cannabigerol (CBG), cannabigerolic acid (CBGA), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), tetrahydrocannabivarinic acid (THCVA), cannabidivarin (CBDV), cannabichromevarin (CBCV), cannabichromevarinic acid (CBCVA), cannabigerovarin (CBGV), cannabichromenic acid (CBCA), cannabidivarinic acid (CBDVA) cannabigerovarinic acid (CBGVA) and cannabigerol monomethyl ether (CBGM), salts thereof, derivatives thereof and mixtures of cannabinoids.

According to one embodiment, the cannabinoid is CBD. According to another embodiment, the cannabinoid is a combination of CBD and CBDA.

According to some embodiments, the content of cannabinoids in the vaporizable formulation is from 20 to 85 wt %, from 25 to 80 wt %, from 30 to 75 wt %, from 35 to 70 wt %, from 40 to 80 wt %, from 45 to 75 wt % or from 50 to 70 wt % of the formulation. According to one embodiment, the cannabinoid is CBD. According to some embodiments, the formulation comprises from 40 to 80 wt % of CBD. According to some embodiments, the formulation comprises from 50 to 80 wt % of CBD. According to some embodiments, the formulation comprises from 60 to 80 wt % of CBD. According to one embodiment, the formulation comprises from 40 to 80 wt % of a combination of CBD and CBDA. According to one embodiment, the formulation comprises from 50 to 80 wt % of a combination of CBD and CBDA. According to one embodiment, the formulation comprises from 60 to 80 wt % of a combination of CBD and CBDA. According to some embodiments, the molar ratio of CBD to CBDA is from 5:1 to 1:5. According to another embodiment, the molar ratio of CBD to CBDA is from 4:1 to 1:4, 3:1 to 1:3, from 2:1 to 1:2 or about 1:1. According to one embodiment, the bisabolol is α-(−)-bisabolol. According to some embodiments, the molar ratio of CBD to CBDA is about 1:1.

According to some embodiments, the content of cannabinoids, e.g. of the CBD, in the formulation is from 1 to 20 wt %, from 2 to 15 wt %, from 3 to 10 wt %, from 1 to 5 wt %, from 5 to 10 wt %, from 10 to 15 wt %, from 10 to 20 wt %, 5 to 25 wt %, from 10 to 25 wt % or from 15 to 20 wt % of the formulation. According to some embodiments, the formulation comprises from 1 to 5 wt % of CBD. According to other embodiments, the vaporizable formulation comprises from 20 to 50 wt %, from 25 to 45 wt %, from 30 to 40 wt %, from 25 to 50 wt %, from 30 to 45 wt % or from 35 to 50 wt % of CBD.

According to some embodiment, the cannabinoid is a combination of CBD and CBDA. According to one embodiment, the vaporizable formulation comprises from 20 to 40 wt % of CBD and from 20 to 40 wt % of CBDA. According to one embodiment, the vaporizable formulation comprises from 25 to 40 wt % of CBD and from 25 to 40 wt % of CBDA. According to one embodiment, the vaporizable formulation comprises from 30 to 40 wt % of CBD and from 30 to 40 wt % of CBDA. According to one embodiment, the vaporizable formulation comprises from 30 to 65 wt % of CBD and from 10 to 55 wt % of CBDA. According to one embodiment, the vaporizable formulation comprises from 30 to 55 wt % of CBD and from 30 to 55 wt % of CBDA. According to one embodiment, the formulation comprises from 35 to 50 wt % of CBD and from 35 to 50 wt % CBDA. According to one embodiment, the vaporizable formulation comprises from 5 to 15 wt % of CBD and from 5 to 15 wt % of CBDA. According to one embodiment, the vaporizable formulation comprises from 3 to 15 wt % or 3 to 10 wt % of CBD and from 3 to 15 wt % or from 3 to 10 wt % of CBDA. According to one embodiment, the vaporizable formulation comprises from 5 to 20 wt % of CBD and from 5 to 20 wt % of CBDA. According to some embodiments, the molar ratio of CBD to CBDA is from 5:1 to 1:5. According to another embodiment, the molar ratio of CBD to CBDA is from 4:1 to 1:4, 3:1 to 1:3, from 2:1 to 1:2 or about 1:1. According to one embodiment, the bisabolol is α-(−)-bisabolol.

According to any one of the above and below embodiments and aspects, the vaporizable formulation is devoid of crystals of cannabinoids. According to some embodiments, the vaporizable formulation comprising CBD is devoid of CBD crystals.

According to any one of the embodiments of the present application, some of the CBD may be replaced by CBDA. According to some embodiment from 10 to 80% of the CBD may be replaced by CBDA. According to some embodiment from 20 to 70% of the CBD is replaced by CBDA. According to some embodiment from 30 to 60% of the CBD is replaced by CBDA. According to some embodiment from 40 to 60% of the CBD is replaced by CBDA. According to some embodiment from 45 to 55% of the CBD is replaced by CBDA. According to some embodiment about 50% of the CBD is replaced by CBDA.

According to one embodiment, the vaporizable formulation comprises from 0.1 to 7 wt %, from 0.2 to 5 wt %, from 0.3 to 3 or from 0.5 to 2 wt % of nicotine or tobacco extract. According to one embodiment, the vaporizable formulation comprises from 0.1 to 7 wt %, from 1 to 4 wt % of nicotine or tobacco extract. According to one embodiment, the vaporizable formulation comprises from 1 to 3 wt % of nicotine or tobacco extract. According to one embodiment, the vaporizable formulation comprises about 0.5, 0.75, 1, 1.25, 1.5, 1.75, 2, 2.25, 2.5, 2.75, 3, 3.25, 3.5, 3.75, or 4 wt % of nicotine or tobacco extract The term “nicotine” refers to the basic form and to a salt of the nicotine such as benzoic acid salt, salicylic acid, sorbic acid, benzoic acid, lauric acid, levulinic acid and malic acid.

According to some embodiments, the vaporizable formulation comprises from 0.2 to 5 wt % nicotine, from 40 to 80 wt % of CBD and from 10 to 50 wt % of the diluent having boiling point of above 140° C. According to other embodiments, the vaporizable formulation comprises from 0.2 to 5 wt % nicotine, from 0.5 to 5 wt % of CBD and from 80 to 95 wt % of the diluent. According to some embodiments, the vaporizable formulation comprises from 0.2 to 5 wt % nicotine, from 5 to 25 wt % of CBD and from 70 to 90 wt % of the diluent. According to some embodiments, the vaporizable formulation comprises from 0.2 to 5 wt % nicotine, from 10 to 20 wt % of CBD and from 70 to 90 wt % of the diluent. According to one embodiment, the vaporizable formulation comprises from 60 to 90 wt %, from 65 to 85 wt %, or from 70 to 85 wt % of the diluent. According to some embodiments, the vaporizable formulation comprises from 0.2 to 5 wt % nicotine, from 20 to 50 wt % of CBD and from 10 to 50 wt % of the diluent. According to other embodiments, the vaporizable formulation comprises from 0.2 to 5 wt % nicotine, from 25 to 45 wt % of CBD and from 80 to 95 wt % of the diluent. According to some embodiments, the vaporizable formulation comprises from 0.2 to 5 wt % nicotine, from 30 to 40 wt % of CBD and from 70 to 90 wt % of the diluent. According to some embodiments, the vaporizable formulation comprises from 0.2 to 5 wt % nicotine, from 25 to 50 wt % of CBD and from 70 to 90 wt % of the diluent. According to one embodiment, the vaporizable formulation comprises from 60 to 90 wt %, from 15 to 40 wt %, or from 70 to 85 wt % of the diluent having. According to some embodiments, the diluent has point of 150° C. or more. According to some embodiments, the diluent has point of 180° C. or more. According to some embodiments, the diluent has point of 200° C. or more. According to some embodiments, the diluent has point of 250° C. or more. According to some embodiments, the diluent has point of at least 290° C. According to some embodiments, the diluent has point of at least 350° C. According to some embodiments, the diluent is glyceryl trioctanoate. According to some embodiments, the diluent is natural bisabolol. According to one embodiment, the diluent is a combination of bisabolol and phytol.

According to some embodiments, the vaporizable formulation comprises from 0.2 to 5 wt % nicotine, from 40 to 85 wt % of CBD and from 10 to 50 wt % of bisabolol such as natural bisabolol. According to some embodiments, the vaporizable formulation comprises from 0.2 to 5 wt % nicotine, from 55 to 85 wt % of CBD and from 20 to 40 wt % of bisabolol such as natural bisabolol. According to some embodiments, from 40 to 60 wt % of CBD is replaced by CBDA. Thus, according to some embodiments, the vaporizable formulation comprises from 0.2 to 5 wt % nicotine, from 20 to 40 wt % of CBD from 20 to 40 wt % of CBDA and from 20 to 40 wt % of bisabolol such as natural bisabolol.

According to some embodiments, the vaporizable formulation comprises from 10 to 25 wt % of CBD, from 70 to 90 wt % of (C8-C12) medium chain triglycerides (MCT), and from 1 to 4 wt % of nicotine. According to some embodiments, the vaporizable formulation comprises from 10 to 25 wt % of CBD, from 70 to 90 wt % of glyceryl trioctanoate, and from 1 to 3 wt % of nicotine. According to some embodiments, the vaporizable formulation comprises from 10 to 20 wt % of CBD, from 75 to 85 wt % of glyceryl trioctanoate, and from 1 to 3 wt % of nicotine. According to some embodiments, the vaporizable formulation comprises from 10 to 20 wt % of CBD, from 75 to 85 wt % of glyceryl trioctanoate, and from 1 to 3 wt % of nicotine and from 0.5 to 2 wt % of benzoic acid. According to some embodiments, from 40 to 60 wt % of CBD is replaced by CBDA. Thus, according to some embodiments, the vaporizable formulation comprises from 5 to 15 wt % of CBD, from 5 to 15 wt % of CBDA, from 70 to 90 wt % of glyceryl trioctanoate, and from 1 to 5 wt % of nicotine.

According to some embodiments, the vaporizable formulation comprises from 55 to 65 wt % of CBD, from 25 to 35 wt % of natural bisabolol, and from 1 to 4 wt % of nicotine. According to some embodiments, the vaporizable formulation comprises from 25 to 35 wt % of CBD, from 25 to 35 wt % of CBDA, from 25 to 35 wt % of natural bisabolol, from 1 to 3 wt % of nicotine. According to some embodiments, the vaporizable formulation comprises about 60 wt % of CBD, about 30 wt % of natural bisabolol, and about 2 wt % of nicotine. According to some embodiments, the vaporizable formulation comprises about 78% of CBD distilled oil, about 30 wt % of natural bisabolol, and about 2 wt % of nicotine.

According to some embodiments, the vaporizable formulation comprises from 55 to 65 wt % of CBD, from 5 to 15 wt % of natural bisabolol, and from 1 to 4 wt % of nicotine. According to some embodiments, the vaporizable formulation comprises from 25 to 35 wt % of CBD, from 25 to 35 wt % of CBDA, from 5 to 15 wt % of natural bisabolol, from 1 to 3 wt % of nicotine. According to some embodiments, the vaporizable formulation comprises about 60 wt % of CBD, about 30 wt % of natural bisabolol, and about 2 wt % of nicotine. According to some embodiments, the vaporizable formulation comprises about 78% of CBD distilled oil, about 30 wt % of natural bisabolol, and about 2 wt % of nicotine. According to some embodiments, the vaporizable formulation comprises about 43% of CBD distilled oil, 43% of CBDA distilled oil about 10-12 wt % of natural bisabolol, and about 2 wt % of nicotine.

According to some embodiments, the vaporizable formulation comprises from 10 to 20 wt % of CBD, from 70 to 90 wt % of natural bisabolol, and from 1 to 4 wt % of nicotine. According to some embodiments, the vaporizable formulation comprises from 5 to 15 wt % of CBD, from 5 to 15 wt % of CBDA, from 70 to 90 wt % of natural bisabolol, and from 1 to 3 wt % of nicotine. According to some embodiments, the vaporizable formulation comprises about 30 wt % of CBD, about from 82 wt % of natural bisabolol, and about 2 wt % of nicotine. According to some embodiments, the formulation further comprises about 1 wt % of natural flavor.

According to some embodiments, the diluent is squalene. According to any one of the above embodiments, the vaporizable formulation further comprises additional excipients such as flavoring agents, antioxidant and preservatives. According to some embodiments, the vaporizable formulation has the content as described in Examples 9-12, 14 and 15, 17-22.

According to any one of the above embodiments, the vaporizable formulation provides a substantially constant dose of CBD and nicotine. According to some embodiments, the deviation between the doses of CBD and of nicotine between two non-consecutive administrations via vaporization is no more than 20%. According to other embodiments, the deviation is no more than 15, 10 or 5%.

According to any one of the above embodiments, the formulation further comprises an antioxidant. According to one embodiment the antioxidant is selected from citric acid, ascorbyl palmitate and a combination thereof According to some embodiments, the antioxidant is selected from citric acid, ascorbyl palmitate, malic acid, tartaric acid oxalic acid, succinic acid and ascorbyl stearate, and any combination thereof. According to some embodiments, the formulation comprises from 0.001 to 1 wt % of the antioxidants, from 0.05 to 0.9, from 0.1 to 0.6 from 0.2 to 0.5 wt % of the antioxidant. According to some embodiments, the formulation comprises from 0.005 to 0.5 wt % of the antioxidant, from 0.01 to 0.4 wt %, from 0.03 to 0.35 wt %, from 0.05 to 0.3 wt % of the antioxidant.

According to another embodiment, the present invention provides a container comprising a vaporizable formulation of the present invention, as defined hereinabove. According to some embodiments, the container comprises from 0.5 to 10 wt % of nicotine, a cannabinoid and at least one diluent having a boiling point of at least 140° C. or at least 200° C. According to another embodiment, the container comprises a vaporizable formulation comprises from 10 to 25 wt % of CBD, from 70 to 90 wt % of (C8-C12) MCT, and from 1 to 3 wt % of nicotine. According to some embodiments, the vaporizable formulation comprises from 10 to 25 wt % of CBD, from 70 to 90 wt % of glyceryl trioctanoate, and from 1 to 3 wt % of nicotine. According to some embodiments, the vaporizable formulation comprises from 10 to 20 wt % of CBD, from 75 to 85 wt % of glyceryl trioctanoate, and from 1 to 3 wt % of nicotine.

According to some embodiments, the container comprises a vaporizable formulation comprising from 55 to 65 wt % of CBD, from 25 to 35 wt % of natural bisabolol, and from 1 to 3 wt % of nicotine. According to some embodiments, the container comprises a vaporizable formulation comprises from 25 to 35 wt % of CBD, from 25 to 35 wt % of CBDA, from 25 to 35 wt % of natural bisabolol, from 1 to 3 wt % of nicotine. According to some embodiments, the container comprises a vaporizable formulation comprises from 25 to 40 wt % of CBD, from 25 to 40 wt % of CBDA, from 5 to 20 wt % of natural bisabolol, from 1 to 3 wt % of nicotine. According to some embodiments, the container comprises a vaporizable formulation comprises about 60 wt % of CBD, about 30 wt % of natural bisabolol, and about 2 wt % of nicotine. According to some embodiments, the container comprises a vaporizable formulation comprises about 78% of CBD distilled oil, about 30 wt % of natural bisabolol, and about 2 wt % of nicotine.

According to some embodiments, the container comprises a vaporizable formulation comprises from 10 to 20 wt % of CBD, from 70 to 90 wt % of natural bisabolol, and from 1 to 3 wt % of nicotine. According to some embodiments, the container comprises a vaporizable formulation comprises from 5 to 15 wt % of CBD, from 5 to 15 wt % of CBDA, from 70 to 90 wt % of natural bisabolol, and from 1 to 3 wt % of nicotine. According to some embodiments, the container comprises a vaporizable formulation comprises about 30 wt % of CBD, about from 82 wt % of natural bisabolol, and about 2 wt % of nicotine. According to some embodiments, the formulation further comprises about 1 wt % of natural flavor.

According to some embodiments, the vaporizable formulation comprises from 5 to 45 wt % of bisabolol, from 40 to 85 wt % of CBD and from 0.5 to 3 wt % of nicotine. According to some embodiments, the vaporizable formulation comprises from 10 to 45 wt % of bisabolol, from 40 to 75 wt % of CBD and from 0.5 to 3 wt % of nicotine. According to some embodiments, the vaporizable formulation comprises from 5 to 45 wt % of bisabolol, from 40 to 75 wt % of CBD and from 0.5 to 3 wt % of nicotine. According to one embodiment, the bisabolol is α-(−)-bisabolol. According to other embodiments, the vaporizable formulation comprises from 5 to 45 wt % of bisabolol, from 50 to 85 wt % of CBD and from 0.5 to 3 wt % of nicotine. According to other embodiments, the vaporizable formulation comprises from 5 to 45 wt % of bisabolol, from 60 to 85 wt % of CBD and from 0.5 to 3 wt % of nicotine. According to some embodiments, the vaporizable formulation comprises from 5 to 45 wt % of bisabolol, from 40 to 85 wt % of CBD and from 0.5 to 3 wt % of nicotine. According to some embodiments, the vaporizable formulation comprises from 5 to 25 wt % of bisabolol, from 60 to 85 wt % of CBD and from 0.5 to 3 wt % of nicotine. According to some embodiments, the vaporizable formulation comprises from 15 to 45 wt % of bisabolol, from 50 to 85 wt % of CBD and from 0.5 to 3 wt % of nicotine. According to other embodiments, the vaporizable formulation comprises from 10 to 25 wt % of bisabolol, from 70 to 85 wt % of CBD and from 0.5 to 3 wt % of nicotine. According to some embodiments, the vaporizable formulation comprises from 15 to 45 wt % of bisabolol, from 70 to 85 wt % of CBD and from 0.5 to 3 wt % of nicotine. According to some embodiments, the vaporizable formulation comprises from 30 to 40 wt % of bisabolol, from 70 to 80 wt %, from 75 to 85 wt %, from 65 to 85 wt %, from 70 to 85 wt %, from 70 to 80, or from 75 to 87 wt % of CBD and from 0.5 to 3 wt % of nicotine. According to some embodiments, the vaporizable formulation comprises from 15 to 35 wt % of bisabolol, from 70 to 80 wt %, from 75 to 85 wt %, from 65 to 85 wt %, from 70 to 85 wt %, from 70 to 80, or from 75 to 87 wt % of CBD and from 0.5 to 3 wt % of nicotine. According to some embodiments, the vaporizable formulation comprises from 5 to 45 wt %, from 10 to 40 wt %, from 15 to 35 wt %, from 20 to 35 wt %, from 5 to 30 wt % or from 5 to 15 wt % of bisabolol and from 0.5 to 3 wt % of nicotine. According to some embodiments, the bisobolol is α-(−)-bisabolol. According to one embodiment, the bisabolol is α-(−)-bisabolol. According to some embodiments, the vaporizable formulation is devoid of CBD crystals. According to some embodiments, the formulation prevents crystallization of CBD.

According to some embodiments, the formulation comprises from 0.001 to 1 wt % of the antioxidants, from 0.05 to 0.9, from 0.1 to 0.6 from 0.2 to 0.5 wt % of the antioxidant. According to some embodiments, the formulation comprises from 0.005 to 0.5 wt % of the antioxidant, from 0.01 to 0.4 wt %, from 0.03 to 0.35 wt %, from 0.05 to 0.3 wt % of the antioxidant. According to any one of the above embodiments, the formulation further comprises an inti-irritant, such as menthol.

According to some embodiments, the vaporizable formulation comprising nicotine is for use in treating nicotine addiction. According to some embodiments, the vaporizable formulation comprising nicotine is for use in treating of smoking addiction. Thus, according to some embodiments, the vaporizable formulation comprising from 0.5 to 10 wt % of nicotine, CBD and at least one diluent having a boiling point of at least 200° C. is for use in treating nicotine and/or smoking addiction. According to some embodiments, vaporizable formulation comprises from 0.2 to 5 wt % nicotine, from 10 to 80 wt % of CBD and from 10 to 50 wt % of the diluent having boiling point of above 250° C. is for use in treating nicotine and/or smoking addiction. According to some embodiments, vaporizable formulation comprises from 0.2 to 5 wt % nicotine, from 10 to 50 wt % of CBD and from 10 to 50 wt % of the diluent having boiling point of above 290° C. is for use in treating nicotine and/or smoking addiction. According to some embodiments, vaporizable formulation comprises from 0.2 to 5 wt % nicotine, from 40 to 80 wt % of CBD and from 10 to 50 wt % of the diluent having boiling point of above 290° C. is for use in treating nicotine and/or smoking addiction.

According to some embodiments, the present invention provides a method of treating a smoking addiction in a subject in need thereof comprising administering the vaporizable formulation of the present invention, as defined in any one of the aspects and embodiments of the present invention. According to some embodiments, the present invention provides a method of treating a nicotine addiction in a subject in need thereof comprising administering the vaporizable formulation of the present invention, as defined in any one of the aspects and embodiments of the present invention.

All terms and definitions used in previous aspects apply herein as well. According to some embodiments, the vaporizable composition is administered by vaporization by using a suitable device for vaporization. According to one embodiment, the administered amount of the composition is from 0.8 to 2 mg/puff.

The method for treatment of nicotine and/or tobacco smoking addiction comprises administering the vaporizable formulation comprising nicotine from 1 to 5 inhalation(s) each time there is a need for a cigarette. According to some embodiments, the method is for treatment of smoking addictions.

According to any one of the above aspects and embodiments, the vaporizable composition may further comprise additional excipients. Non-limiting examples of such excipients are ethanol, ethyl acetate, propylene glycol, glycerol, tocopherol, 1,3-propane diol, MCT, coconut oil, triethyl citrate, and squalane. According to some embodiments, the additional excipients may be natural or artificial flavoring agents and antioxidants. According to some embodiments, the vaporizable composition may further comprise additional terpenes, such as menthol used, for examples as flavoring agents. According to some embodiments, the formulation may be devoid of the excipients listed above in the paragraph. According to some embodiments, the formulation is devoid of the one, some or all of the excipients listed above in the paragraph.

According to any one of the aspects and embodiments of the present invention, all the compounds such as diluents and excipients are generally considered as safe (GRAS).

The terms “a,” “an,” or “the” as used herein include plural referents unless the context clearly dictates otherwise.

The term “comprising” always includes the term “consisting” and when the term “comprising” appears in an embodiment of the disclosure, this same embodiment wherein the term “consisting” replaces the term “comprising” is always also an embodiment of the disclosure.

As used herein, the term “about”, when referring to a measurable value such as an amount, a temporal duration, and the like, is meant to encompass variations of +/−10%, or +/−5%, +/−1%, or even +/−0.1% from the specified value.

According to some embodiments, the term “about 0 wt %” refer to 0.0001 wt %, 0.001 wt %, 0.01 wt % or 0.1 wt % or that the compound is absent.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “no more” and “less than” may, in some embodiments, be used interchangeably.

Having now generally described the invention, the same will be more readily understood through reference to the following examples, which are provided by way of illustration and are not intended to be limiting of the present invention.

### EXAMPLES

### Materials and Methods

### Ceramic Cartridges

The vaping ceramic cartridges used in the test were cartridges from VapePod connected to a 3.2 V battery. For each inhalation the battery was breath activated for a duration of 1.5 sec. The cartridge resistance was of 1.35Ω in examples 1,2,4,6,7, 8 and 1.1Ω in examples 5,9,10 and 11.

### Simulation of Inhalation

Simulation of inhalation was performed by aspiration from the ceramic vape cartridge at a rate of 1 L/min (17 mL/sec.) for three seconds. The aspiration was performed by the aim of a peristaltic pump with silicon tubing. The mouthpiece of the cartridge was connected to the inlet side of the peristaltic pump tube. This operation of aspiration for three seconds is referred as “inhalation”.

In all experiments, unless stated explicitly, natural bisabolol was used.

For consistency tests, the full cartridge connected to the battery is weighed. The cartridge mouthpiece was connected to the inlet tube of the peristaltic pump. Ten successive inhalations were performed at intervals of 30 seconds. The cartridge with the battery was weighed again. The weight difference is divided by 10 and the value obtained represents the average amount released for each inhalation. The operation was repeated until partial or total emptying of the cartridge. The process started with a fully charged battery. The battery was changed or recharged after 80 inhalations to ensure operation with constant power voltage.

### Example 1. Flavor and Irritation of THC-Rich Formulation Comprising Different Diluents

The cartridges were filled with 0.5 mL of THC-rich, distilled, cannabis extract without diluent or with 15% of diluent, as detailed in Table 1. The THC-rich extract comprises 85% of THC and about 5% sesquiterpenes (detailed list of sesquiterpenes is provided in Table 2). Five volunteers made three inhalations from a cartridge and reported their feeling in relation to taste and irritation. The results are presented in Table 1.

TABLE 1
Experimental arrangement and results

| Diluent | Flavor | Irritation |
|---|---|---|
| No diluent | 1 | 3 |
| 15% Synthetic bisabolol | 4 | 2 |
| 14% Synthetic bisabolol + 1% menthol | 3 | 1 |
| 15% Natural bisabolol | 2 | 2 |
| 15% phytol | 2 | 4 |
| 7.5% Natural bisabolol + 7.5% phytol | 1 | 1 |

Flavor: 1 good or very good, 2 almost good, 3 medium, 4 almost bad, 5 bad or very bad
Irritation: 1 low, 3 medium, 5 strong

TABLE 2
Sesquiterpenes in the THC-rich oil

| Identified Compound | Content |
|---|---|
| Beta-Caryophylene | 1.02% |
| α-Caryophylene | 0.32% |
| Eudesma-4(14),11-diene | 0.23% |
| Chamigrene | 0.27% |
| Guaia-3,9-diene or | 0.28% |
| Eudesma-3,7(11)-diene | 0.23% |
| Bisabolol | 0.42% |
| Others | 2% |
| Total | 4.77% |

Interestingly, 15 wt % of phytol in fact worsened the flavor and the irritation of the THC oil. A combination of natural bisabolol and phytol in cumulative concentration of 15 wt % provided the best effect with respect to flavor and feeling.

### Example 2. THC-Rich Formulations Prepared with Different Diluents

The cartridge was filled with THC-rich, distilled, cannabis extract (as in Example 1) with or without diluent, as described in Table 3. The change of the dose while emptying the cartridge was measured as described in materials and methods.

TABLE 3
THC-rich distilled extract formulated with different diluents

| Combination | THC-rich | Diluent type |
|---|---|---|
| No. | extract content | and content |
| 1 | 100% | — |
| 2 | 80% | propylene glycol (PG)-20% |
| 3 | 50% | *PG-50% |
| 4 | 80% | medium chain triglycerides |
| | | (MCT)-20% |
| 5 | 85% | 7.5% natural bisabolol and |
| | | 7.5% phytol. |

*resistance of the cartridge was 1.1 Ω instead of 1.35 Ω

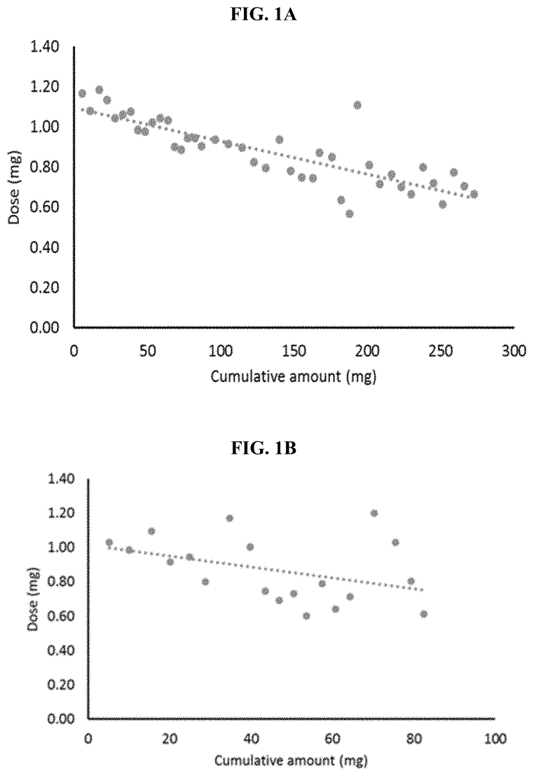

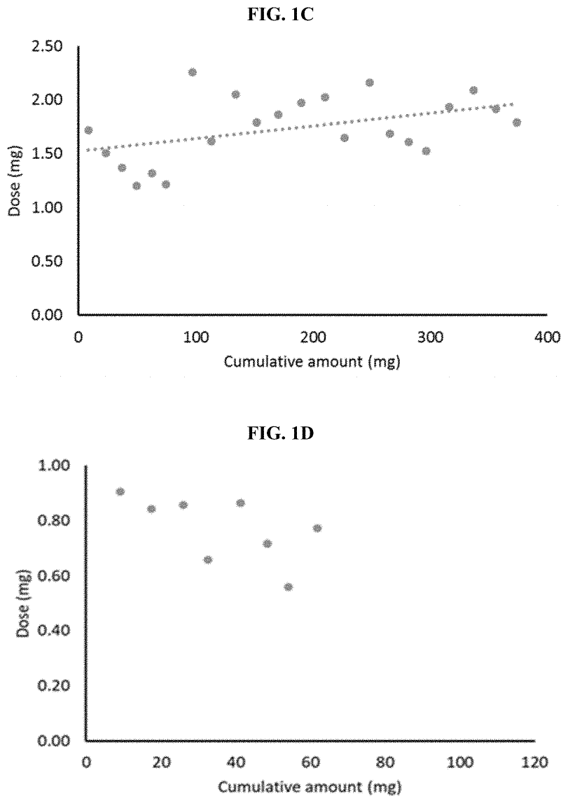

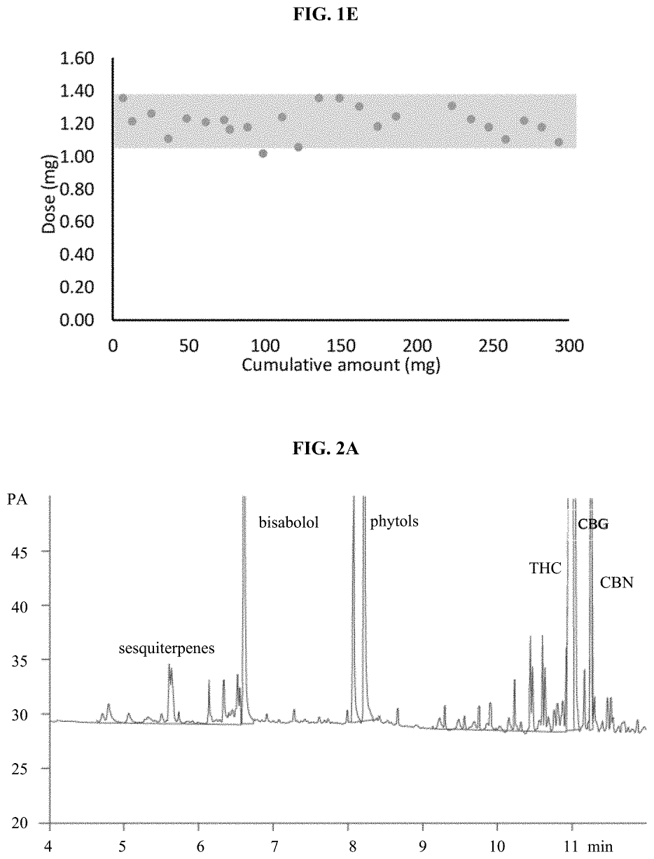

The results are presented in FIG. 1A (combination 1), FIG. 1B (combination 2), FIG. 1C (combination 3), FIG. 1D (combination 4), and FIG. 1E (combination 5).

As can be seen from the FIG. 1A, there was a progressive decrease of the dose while emptying the cartridge, when a composition without a diluent was used. Combinations 2 and 3 provided a pronounced dose fluctuation (from 0.6 and 1.2 mg per dose for combination 2 and from 1.1 to 2.3 mg per dose for combination 3). The inhaled dose using composition 4 was chaotic and decreased with emptying the cartridge. Contrary to all above combination, composition comprising 85% THC-rich distilled extract, 7.5% bisabolol and 7.5% phytol provided practically constant dose of 1.2 mg of the composition.

Combining all said data it is clear that composition comprising 85% THC-rich distilled extract, 7.5% bisabolol and 7.5% phytol provided practically constant dose upon inhalation. This dose did not change upon emptying the cartridge. The combination also showed good flavor properties and did not cause to irritation typical to inhalation of cannabinoid concentrate.

### Example 3. Stability of CBD Formulations with Different Diluents

5 g of the following different blends were prepared. The blends contained a CBD distillate containing 92% of CBD, a diluent at different concentrations and 1% natural flavor (Girl School cookies from Floraplex—USA). The diluent was phytol, natural bisabolol or a combination of phytol and natural bisabolol. 2 mg of crystal seed were added to the vial to initiate crystallization. The vials were stored at 22° C. The vials were checked after a week to see if there is crystallization. The results are presented in the Table 4.

TABLE 4
Effect of the diluent and its concentration on crystallization of CBD

| | % diluent in blend | | | | |
|---|---|---|---|---|---|
| Diluent | 28 | 32 | 36 | 40 | 44 |
| Phytol | + | + | + | + | − |
| Phytol/bisabolol 75:25 | + | + | − | − | − |
| Phytol/bisabolol 50:50 | + | + | − | − | − |
| Bisabolol | + | − | − | | |

+crystallization, −no crystallization

It can be seen that when only phytol was used as a sole diluent it required about 44% of phytol to prevent crystallization. Obvious that the concentration of CBD in such composition is about 50%. When the combination of phytol+bisabolol was used much lower concentration of diluent was required—about 36% of diluent to stabilize CBD and to prevent crystallization (upon addition of crystallization seeds). It is also clear that this combination stabilizes much higher concentrations of CBD—about 60%. When bisabolol alone was used, the concentration of about 30% or more was sufficient to prevent crystallization upon addition of crystallization seeds.

Further, cartridges with the same formulations were prepared (without seed enhancing crystallization). The cartridges were tested for taste by five volunteers. The results are presented in Table 5.

TABLE 5
Taste of compositions comprising CBD and different diluents

| | % diluent in blend | | | | |
|---|---|---|---|---|---|
| Diluent | 28 | 32 | 36 | 40 | 44 |
| Phytol | NA | NA | NA | NA | 1 |
| Phytol/bisabolol 75:25 | NA | NA | 1 | 2 | 2 |
| Phytol/bisabolol 50:50 | NA | NA | 1 | 2 | 2 |
| Bisabolol | 1 | 2 | 3 | | |

Flavor: 1 good or very good, 2 almost good, 3 medium, 4 almost bad, 5 bad or very bad, NA—not available-samples were not tested due to crystallization

The best flavor was obtained for the blend comprising 36% of the combination of phytol/bisabolol with percent ratio of 75:25 or 50:50 and for bisabolol alone 28%. As can be seen from Table 4, this combination also provides highly stable composition. None of the formulation described in Table 5 caused to any irritation.

### Example 4. The Content of the Initial Formulation and Aerosol Obtained Upon its Vaporization

We tested the content of the aerosol obtained upon vaporization of the formulations comprising THC-rich, distilled, extract by gas chromatography. The results of a comparison of the initial composition of the cartridge, the composition of the aerosol for the first inhalations (referred as “Initial aerosol”) and for inhalations after emptying of most of the cartridge content (referred as “Final aerosol”), are provided in Table 6.

TABLE 6
Content of THC-rich oil formulations before and after vaporization

| | Other | | Synthetic | Others | |
|---|---|---|---|---|---|
| | Terpenes | Bisabolol | phytol | Cannabinoids | THC |
| | (%) | (%) | (%) | (%) | (%) |
| Cartridge initial | 4.9 | 6.7 | 6.0 | 10.1 | 72.3 |
| composition | | | | | |
| Initial aerosol | 4.6 | 7.8 | 6.5 | 8.3 | 72.8 |
| Final aerosol | 5.3 | 8.5 | 7.5 | 9.4 | 69.3 |

It can be easily seen that the composition of the aerosol is practically the same as that in the cartridge and that both at the beginning and the end of the use of this cartridge.

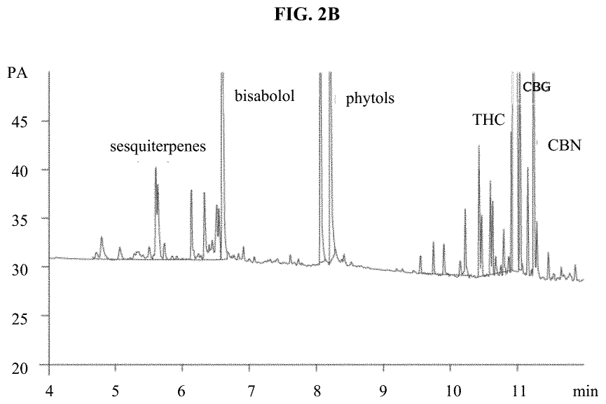

The chromatograms of the THC-rich formulation comprising bisabolol and synthetic phytol in cartridge and in the vaporized aerosol is presented in FIG. 2. No new peak was observed in the aerosol, indicating that that there was no sensible amount of decomposition of the product. It can be clearly concluded that composition comprising about 6-8 wt % of bisabolol and about 6-8 wt % of phytol provides substantially constant dose of THC upon multiple inhalations of the same cartridge.

### Example 5. Stability of Oversaturated CBD Solution

Vials were prepared with 5 ml of oversaturated blend of CBD with different diluent. The blend was composed of CBD distilled oil (containing 90% CBD and less than 1% bisabolol) or CBD isolate (more than 99% CBD), and natural bisabolol and phytol. The stability of the solution to crystallization in the vial after a week of storage at either 10 or 22° C. is presented in Table 7.

TABLE 7
Stability of oversaturated CBD blend to crystallization

| CBD source | Diluent | % diluent in blend | Crystallization |
|---|---|---|---|
| CBD 90% | Natural | 5% | − |
| | Bisabolol | 10% | − |
| | | 20% | − |
| | Phytol | 15% | + |
| CBD isolate | Natural | 10% | + |
| | Bisabolol | 20% | − |

+crystallization, −no crystallization

As follows from this Example, 5% of natural bisabolol could efficiently prevent crystallization of CBD in formulation comprising 85.5 wt % of CBD. In comparison 15 wt % of phytol did not prevent crystallization in formulation containing 76.5% CBD. Same results were obtained for vials maintained at 10 and 22° C.

### Example 6. Formulations Comprising CBD and CBDA

The following composition was prepared:
45% distilled CBD extract with 90% CBD
40% CBDA crystals
7% of natural bisabolol
7% of phytol
1% of natural flavor (Girl School cookies from Floraplex—USA).

No crystallization was observed even with seeding.

Cartridge was prepared with this formulation and tested by three volunteers. The taste was very good and there was no irritation. Typically, during heating, CBDA is decarboxylated to CBD and CO₂. The total amount of CBD (CBD and decarboxilated CBDA) in the composition was about 75%. In comparison, in the optimal condition without CBDA (Table 4) is about 60%. The advantage is higher potency but also lower amount of diluent—15% instead of about 30%. The source of CBDA may be CBDA crystal or CBDA from extract. Similar example was performed using 7.5 wt % of natural bisabolol and 7.5 wt % of phytol and similar results were obtained.

### Example 7. Compositions of Bisabolol and Additional Sesquiterpenes

5 g of the following different compositions were prepared. The composition contained a CBD distillate containing 85% of CBD, a diluent and 1% natural flavor additive (Girl School cookies from Floraplex—USA). The diluent was natural bisabolol, or bisabolol together with a blend of several natural sesquiterpenes. The blend was composed of 20% valenciene, 20% humulene, 20% cadinene, 20% caryophyllene, and 20% caryophyllene oxide. The formulations contained 0.1% citric acid as and 0.05% of ascorbyl palmitate as an antioxidant. That permits to limit coloration of the oil in the cartridge. The content of the bisabolol and or the blend is provided in Table 8.

The cartridges were tested for taste by five volunteers. The results are presented in Table 8. Replacement of a part of the bisabolol by 5% of the sesquiterpene blend does not affect the flavor. Increase to 15% or 22% has a negative effect on the flavor.

TABLE 8
Flavor of compositions
comprising CBD and different diluents compositions

| Content in formulation (%) | | | | Flavor |
|---|---|---|---|---|
| CBD | Flavor additive | Bisabolol | Sesquiterpene blend | score |
| 71 | 1 | 28 | 0 | 1 |
| 71 | 1 | 23 | 5 | 1 |
| 71 | 1 | 13 | 15 | 2 |
| 71 | 1 | 5 | 22 | 3 |

Flavor: 1 good or very good, 2 almost good, 3 medium, 4 almost bad, 5 bad or very bad

### Example 8. Formulations Comprising Nicotine and their Use in Treatment of Smoking Addiction

A formulation was prepared which contains:
6.8 g CBD distilled oil containing 88% CBD (˜60 wt % CBD in formulation)
3.0 g natural bisabolol (30 wt %)
0.2 g nicotine (2 wt %)
   Cartridges were prepared with this formulation.

Ten volunteers who used to smoke between 10 and 15 cigarettes per day participated in the test for one week. These volunteers unsuccessfully tried in the past to stop smoking. They tried to replace smoking by e-cigarette vaping without success. They were instructed a) to try to limit cigarette consumption and b) to make a cycle of three to five inhalations of the CBD/nicotine cartridge each time they need a cigarette. The experiment was successful for three out of the five volunteers. They made a cycle of inhalations between 5 and 10 times per day for a period of one week. During this period, they reduced their consumption of cigarettes by about 70% and did not experience physical or psychological withdrawal effects. Their general feeling was better than in case of use of regular e-cigarette with nicotine (but without CBD).

### Example 9

A formulation was prepared which contains:
6.8 g CBD distilled oil containing 88% CBD (˜60 wt % CBD in formulation)
1.5 g natural bisabolol and 1.5 g phytol (30 wt %)
0.2 g nicotine (2 wt %)

Cartridges were prepared with this formulation and tested by three volunteers. The results were essentially similar to those of the Example 8. During this period, the participants reduced their consumption of cigarettes by about 70% and did not experience any physical or psychological withdrawal effects.

### Example 10

A formulation was prepared which contains:
1.5 g CBD isolate
8.2 g glyceryl trioctanoate
0.1 g of 1% of natural flavor (Girl School cookies from Floraplex—USA).
0.2 g nicotine (2 wt %)
   Cartridges were prepared with this formulation.

Ten volunteers who used to smoke between 10 and 15 cigarettes per day participated in the test for one week. These volunteers unsuccessfully tried in the past to stop smoking, for example replacing smoking by e-cigarette vaping. They were instructed to make a cycle of three inhalations of the formulation cartridge each time they need a cigarette. If after this cycle of three inhalation they still have a need for a regular cigarette, they were allowed to smoke a regular cigarette.

The consumption of cigarettes during this period is presented in the Table 9. The test was successful for seven out of ten volunteers. For five volunteers, there was total cessation of cigarette smoking and for two volunteers the cigarette reduction was of more than 80%. In addition, the amount of nicotine inhaled with the method used is low. The global daily amount of nicotine in vaping aerosol was of about 0.6 mg to be compared to about 15 mg in regular vaping or in smoking of 10 cigarettes. This small amount combined with CBD is sufficient to obtain a drastic reduction in the amount of cigarette smoked.

TABLE 9
Cigarette consumption before and during the test

| | Number of | Daily average amount | |
|---|---|---|---|
| | inhalations | of cigarette smoked | |
| Volunteer | cycles per days | Before the test | During the test |
| 1 | 10 | 11 | 0 |
| 2 | 8 | 12 | 0 |
| 3 | 6 | 10 | 10 |
| 4 | 8.5 | 15 | 5 |
| 5 | 9 | 11 | 1 |
| 6 | 11 | 10 | 2 |
| 7 | 5 | 11 | 10 |
| 8 | 9 | 13 | 0 |
| 9 | 9.5 | 10 | 0 |
| 10 | 9 | 8 | 0 |

### Example 11

A formulation was prepared which contains:
1.5 g CBD isolate
8.05 g glyceryl trioctanoate
0.1 g of 1% of natural flavor (Girl School cookies from Floraplex—USA).
0.2 g nicotine (2 wt %)
0.15 g of benzoic acid

Cartridges were prepared with this formulation. Three volunteers tested the formulations with a protocol similar to that of Example 10. The three volunteers reduced their consumption of cigarette by more than 50% during the period of the test.

### Example 12

A formulation was prepared which contains:
1.5 g CBD isolate
8.2 g of squalane
0.1 g of 1% of natural flavor (Girl School cookies from Floraplex—USA).
0.2 g nicotine (2 wt %)

Cartridges were prepared with this formulation. Three volunteers tested the formulations with a protocol similar to that of Example 10. The three volunteers reduced their consumption of cigarette by more than 50% during the period of the test.

### Example 13. The Content of the Initial Formulation and Aerosol Obtained Upon its Vaporization

We tested the content of the aerosol obtained upon vaporization of the formulations comprising the formulation of Example 10 by gas chromatography. The results of a comparison of the initial composition of the cartridge, the composition of the aerosol for the first inhalations (referred as “Initial aerosol”) and for inhalations after emptying of most of the cartridge content (referred as “Final aerosol”), are provided in Table 10.

TABLE 10
Content of nicotine/CBD formulation before and after vaporization

| | Glyceryl trioctonate | CBD | Nicotine |
|---|---|---|---|
| | (%) | (%) | (%) |
| Cartridge initial | 82 | 15 | 2 |
| composition | | | |
| Initial aerosol | 81.5 | 14.5 | 2.1 |
| Final aerosol | 82.5 | 13.8 | 1.9 |

It can be easily seen that the composition of the aerosol is practically the same as that in the cartridge and that both at the beginning and the end of the use of this cartridge.

### Example 14

A formulation was prepared which contains:
2.5 g CBD isolate
7.2 g glyceryl trioctanoate
0.1 g of 1% of natural flavor (Girl School cookies from Floraplex—USA).
0.2 g nicotine (2 wt %)

Cartridges are prepared with this formulation. A volunteer tested the formulations with a protocol similar to that of Example 10. He reduced his tobacco consumption by 60% during the period of the test.

### Example 15

A formulation was prepared which contains:
4 g CBD isolate
5.7 g glyceryl trioctanoate
0.1 g of 1% of natural flavor (Girl School cookies from Floraplex—USA).
0.2 g nicotine (2 wt %)

Cartridges are prepared with this formulation. A volunteer tested the formulations with a protocol similar to that of Example 10. He reduced its tobacco consumption by 45% during the period of the test.

### Example 16

A volunteer who was used to consume 0.35 mL of e-liquid containing 5% nicotine per day used the formulation of Example 10 with the protocol of this Example for a week. He reduced his consumption of e-liquid by 55% during the period of the test. During this period, he felt quiet with reduced anxiety and insomnia.

### Example 17

A formulation was prepared which contains:
0.75 g CBD isolate (containing about 85% CBD)
0.75 g CBDA isolate (containing about 85% CBDA)
8.2 g glyceryl trioctanoate
0.1 g of 1% of natural flavor (Girl School cookies from Floraplex—USA).
0.2 g nicotine (2 wt %)
   Cartridges were prepared with this formulation.

### Example 18

A formulation are prepared which contains:
0.75 g CBD isolate
0.75 g CBDA
8.3 g glyceryl trioctanoate
0.1 g of 1% of natural flavor (Girl School cookies from Floraplex—USA).
0.1 g nicotine (1 wt %)
   Cartridges are prepared with this formulation.

### Example 19

A formulation are prepared which contains:
0.75 g CBD isolate
0.75 g CBDA
8.1 g glyceryl trioctanoate
0.1 g of 1% of natural flavor (Girl School cookies from Floraplex—USA).
0.3 g nicotine (3 wt %)
   Cartridges are prepared with this formulation.

### Example 20

A formulation are prepared which contains:
43 g CBD isolate
43 g CBDA
11 g alpha-bisabolol
3 g nicotine (3 wt %)
   Cartridges are prepared with this formulation.

A formulation are prepared which contains:
43 g CBD isolate
43 g CBDA
13.3 g alpha-bisabolol
0.7 g of a mix of terpenes.
   Cartridges are prepared with this formulation.

### Example 21

A formulation are prepared which contains:
43 g CBD isolate
43 g CBDA
10 g alpha-bisabolol
1 g of 1% of natural flavor (Girl School cookies from Floraplex—USA).
3 g nicotine (3 wt %)
   Cartridges are prepared with this formulation.

### Example 22

A formulation are prepared which contains:
43 g CBD isolate (containing about 85% CBD)
43 g CBDA isolate (containing about 85% CBDA)
12 g alpha-bisabolol
2 g nicotine (3 wt %)
   Cartridges are prepared with this formulation.

Ten volunteers who are used to smoke between 10 and 15 cigarettes per day participate in the test for one week. These volunteers unsuccessfully tried in the past to stop smoking, for example replacing smoking by e-cigarette vaping. They are instructed to make a cycle of three inhalations of the formulation cartridge each time they need a cigarette. If after this cycle of three inhalation they still have a need for a regular cigarette, they are allowed to smoke a regular cigarette.

The test is successful for more than 50% of the volunteers. The global daily amount of nicotine in vaping aerosol is of about 0.6 mg to be compared to about 15 mg in regular vaping or in smoking of 10 cigarettes. This small amount combined with CBD is sufficient to obtain a drastic reduction in the amount of cigarette smoked.

Although the present invention has been described herein above by way of preferred embodiments thereof, it can be modified, without departing from the spirit and nature of the subject invention as defined in the appended claims

1. A vaporizable formulation comprising (i) a cannabinoid, (ii) from 0.5 to 10 wt % of nicotine, and (iii) at least one diluent, wherein the cannabinoid is selected from CBD, CBDA or a combination thereof.

2. The vaporizable formulation according to claim 1, wherein the least one diluent has a boiling point of at least 140° C., at least 200° C., at least 290° C. or at least 350° C.

3. The vaporizable formulation according to claim 1, wherein the least one diluent is selected from the group consisting of natural bisabolol, sesquiterpenes, diterpenes, medium chain triglycerides (MCT), glyceryl trioctanoate, glyceryl tridecanoate, squalane, triethyl citrate, PEG and any combination thereof.

4. The vaporizable formulation according to claim 1, comprising from 50 to 90 wt % of the diluent.

5. The vaporizable formulation according to claim 4, wherein the diluent is (C8-C12) medium chain triglycerides.

6. The vaporizable formulation according to claim 5, wherein the diluent is glyceryl trioctanoate.

7. The vaporizable formulation according to claim 4, comprising from 5 to 25 wt % of the cannabinoid.

8. The vaporizable formulation according to claim 1, comprising from 5 to 50 wt % of the diluent.

9. The vaporizable formulation according to claim 1, comprising from 5 to 20 wt % of the diluent.

10. The vaporizable formulation according to claim 8, wherein the diluent is natural bisabolol.

11. The vaporizable formulation according to claim 8, comprising from 50 to 90 wt % of the cannabinoid.

12. The vaporizable formulation according to claim 1, wherein the cannabinoid is CBD.

13. The vaporizable formulation according to claim 1, wherein the cannabinoid is a combination of CBD and CBDA.

14. The vaporizable formulation according to claim 13, wherein the molar ratio of CBD to CBDA is from 5:1 to 1:5, from 2:1 to 2:1 or about 1:1.

15. The vaporizable formulation according to claim 1, comprising from 1 to 4 wt % of nicotine.

16. The vaporizable formulation according to claim 1, comprising (i) from 60 to 80 wt % of the cannabinoid from 5 to 20 wt % of natural bisabolol, and from 1 to 4 wt % of nicotine, (i) from 50 to 70 wt % of the cannabinoid from 20 to 40 wt % of natural bisabolol, and from 1 to 4 wt % of nicotine, (ii) from 10 to 30 wt % of the cannabinoid, from 60 to 90 wt % of (C8-C12) medium chain triglycerides (MCT), and from 1 to 4 wt % of nicotine, or (iii) from 10 to 30 wt % of the cannabinoid, from 60 to 90 wt % of glyceryl trioctanoate, and from 1 to 4 wt % of nicotine, wherein the cannabinoid is selected from CBD and a combination of CBD and CBDA.

17. The vaporizable formulation according to claim 16, wherein the molar ratio of CBD to CBDA is from 2:1 to 1:2 or about 1:1

18. A method of treating a nicotine and/or tobacco smoking addiction in a subject in need thereof comprising administering the vaporizable formulation according to claim 1.
